(19) United States
(12) Patent Application Publication
(10) Pub. No.: US 2009/0325889 A1
(43) Pub. Date: Dec. 31, 2009

(54) HEPATITIS C SERINE PROTEASE INHIBITORS AND USES THEREFOR

(76) Inventors: David Alan Campbell, San Diego, CA (US); David T. Winn, San Diego, CA (US); Juan Manuel Betancort, San Diego, CA (US); Michael E. Hepperle, San Diego, CA (US)

Correspondence Address: SCHWEGMAN, LUNDBERG & WOESSNER, P.A., P.O. BOX 2938, MINNEAPOLIS, MN, 55402, US

(21) Appl. No.: 11/996,280

(22) PCT Filed: Jul. 28, 2006
(86) PCT No.: PCT/US06/29708

§ 371 Date: Jul. 22, 2008

Related U.S. Application Data
(60) Provisional application No. 60/704,355, filed on Aug. 1, 2005.
(60) Provisional application No. 60/728,472, filed on Oct. 20, 2005.

(51) Int. Cl. A61K 38/06; C12N 9/99; A61K 38/05; C07K 5/083; C07F 5/02; A61P 31/12

(52) U.S. Cl. 514/18; 435/184; 514/19; 530/331; 546/13; 549/4; 549/213; 564/8

(57) ABSTRACT

The present invention provides novel compounds which mimic peptides with a C-terminal penultimate proline, such compounds being useful as protease inhibitors, particularly as inhibitors of serine proteases, and more particularly as inhibitors of the NS3 serine protease from hepatitis C. The compounds find utility as antiviral agents directed at hepatitis C. The invention further provides methods of employing such inhibitors, alone or in combination with other therapeutic agents, to treat hepatitis C infection in a subject in need of such treatment.

### FIELD OF THE INVENTION

The present invention relates to novel compounds that are useful as protease inhibitors, particularly as inhibitors of serine proteases, and more particularly as inhibitors of the NS3 serine protease and its associated co-factor, NS4a from hepatitis C. Because these inhibitors interfere with protease activity necessary for hepatitis C survival, the compounds find utility as antiviral agents directed at people infected with hepatitis C virus. The invention further relates to methods of employing such inhibitors, alone or in combination with other therapeutic agents, to treat hepatitis C infection in a subject in need of such treatment.

### BACKGROUND OF THE INVENTION

Hepatitis C virus (“HCV”) is the causative agent for hepatitis C, a chronic infection characterized by jaundice, fatigue, abdominal pain, loss of appetite, nausea, and darkening of the urine. HCV, belonging to the hepacivirus genus of the Flaviviriae family, is an enveloped, single-stranded positive-sense RNA-containing virus. The long-term effects of hepatitis C infection as a percentage of infected subjects include chronic infection (55-85%), chronic liver disease (70%), and death (1-5%). Furthermore, HCV is the leading indication for liver transplant. In chronic infection, there usually presents progressively worsening liver inflammation, which often leads to more severe disease states such as cirrhosis and hepatocellular carcinoma.

The HCV genome (Choo et al., Science 1989, 244, 359-362; Simmonds et al., Hepatology 1995, 21, 570-583) is a highly variable sequence exemplified by GenBank accession NC_{—}004102 as a 9646 base pair single-stranded RNA comprising the following constituents at the parenthetically indicated positions: 5′NTR (i.e., non-transcribed region) (1-341); core protein (i.e., viral capsid protein involved in diverse processes including viral morphogenesis or regulation of host gene expression) (342-914); E1 protein (i.e., viral envelope) (915-1490); E2 protein (i.e., viral envelope) (1491-2579); p7 protein (2580-2768); NS2 protein (i.e., non-structural protein 2) (2769-3419); NS3 protease (3420-5312); NS4a protein (5313-5474); NS4b protein (5475-6257); NS5a protein (6258-7601); NS5b RNA-dependent RNA polymerase (7602-9372); and 3′NTR (9375-9646). Additionally, a 17-kDalton −2/+1 frameshift protein, “protein F”, comprising the joining of positions (342-369) with (371-828) may provide functionality originally ascribed to the core protein.

The NS3 (i.e., non-structural protein 3) protein of HCV has serine protease activity, the N-terminal of which is produced by the action of a NS2-NS3 metal-dependent protease, and the C-terminal of which is produced by auto-proteolysis. Genotypes including but not limited to 1a, 1b, 2 and 3 and mutant forms of the NS3 protein are also known and behave in a similar fashion. See, for example, M. Yi, et al., J. Biol. Chem., 281, 8205 (2006) first published Dec. 12, 2005, as well as the references cited therein. The HCV NS3 serine protease, its mutants and its associated cofactor, NS4a, process all of the other non-structural viral proteins of HCV. Accordingly, the HCV NS3 protease, or a mutant forms thereof where applicable, is essential for viral replication.

Several compounds have been shown to inhibit the hepatitis C serine protease, but all of these have limitations in relation to the potency, stability, selectivity, toxicity, and/or pharmacodynamic properties. Such compounds have been disclosed, for example, in published U.S. patent applications 2004/0266731, 2002/0032175, 2005/0137139, 2005/0119189, and 2004/9977600A1, and in published PCT patent application WO 2005/035525. Accordingly, a need exists for compounds that are useful for inhibiting the serine protease of HCV.

### SUMMARY OF THE INVENTION

In accordance with one aspect of the present invention, there are provided compounds that are effective in inhibiting proteases, particularly serine proteases, and more particularly the HCV NS3 serine protease. These compounds can be used alone or as constituents of compositions provided by the invention to inhibit the processing of non-structural proteins necessary in the HCV life cycle, including without limitation the NS2, NS3, NS4a, NS4b, and NS5a proteins, and the NS5b RNA-dependent polymerase. The present 5 invention also provides methods for inhibiting proteases, particularly serine proteases, and more particularly HCV NS3 serine protease.

### DETAILED DESCRIPTION OF THE INVENTION

In accordance with one aspect, the present invention provides compounds of Formula I:

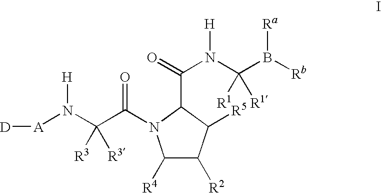

and stereoisomers, solvates, hydrates, tautomers, prodrugs, pharmaceutically acceptable salts, and mixtures thereof,

wherein:
A is a bond, —C(O)—, —C(O)O—, —C(O)NH—, —S(O)—, or —S(O)₂—;
D is —R⁶, -alkyl-R⁶, -alkenyl-R⁶, -alkynyl-R⁶, —OR⁶, —NHR⁶, —N(R⁶)₂, or —C(O)R⁶;
R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
when R¹, R¹′ and R⁴ are not hydrogen, then
   R is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
when R¹ and R¹′ are not hydrogen, and R⁴ is hydrogen, then
   R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷′, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH2)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
when either R¹ or R¹′ is hydrogen, and R⁴ is not hydrogen, then
   R² is —(CH₂)ₓ—R⁸′, —(CH₂)ₓ—NH(R⁹)—R⁸, —(CH₂)_{w}—O—R⁸′, —(CH₂)ₓ—S—R⁸, —O—(CH₂)_{w}—R⁸′, —NH(R⁹)—(CH₂)ₓ—R⁸′, —S—(CH₂)ₓ—R⁸, —O—R⁸′, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸;
when either R¹ or R¹′ is hydrogen, and R⁴ is hydrogen, then
   R² is —(CH₂)ₓ—R⁸′, —(CH₂)ₓ—NH(R⁹)—R⁸, —(CH₂)_{w}—O—R⁸′, —(CH₂)ₓ—S—R⁸, —O—(CH₂)_{w}—R⁸′, —NH(R⁹)—(CH₂)ₓ—R⁸′, —S—(CH₂)ₓ—R⁸, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸;
   R⁶ is a substituted or unsubstituted aryl, heteroaryl, cycloalkyl, heterocyclyl, cycloalkylidenyl, or heterocycloalkylidenyl group;
R⁷ and R¹¹ are independently a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁷′ is a substituted or unsubstituted alkyl, cycloalkyl, cycloalkenyl, cycloalkylalkyl, cycloalkenylalkyl, saturated or unsaturated heterocyclyl, saturated or unsaturated heterocyclylalkyl group, or an aryl or heteroaryl group substituted with NR¹⁰R¹¹;
R⁸ is a divalent substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R⁸′ is a divalent substituted or unsubstituted cycloalkyl, cycloalkenyl, cycloalkylalkyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R⁹ and R¹⁰ are independently hydrogen or R⁷;
w is in the range 1-6; and
x is in the range 0-6.

In some embodiments of compounds of Formula I, Rₐ and R_{b} are independently hydroxyl, methoxy, ethoxy, n-propoxy, i-propoxy, or n-butoxy; or together are 1,2-dioxaethylene, 1,3-dioxapropylene, 1,3-dioxapropylene, 2,3-dimethyl-2,3-dioxabutane, or pinanedioxy.

In some embodiments of compounds of Formula I, R¹ and R¹′ are independently hydrogen or a substituted or unsubstituted alkyl group.

In another aspect, the present invention provides compounds of Formula I

wherein:
R¹ and R¹′ are independently a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R⁴ is a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group; and
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷.

In another embodiment of the invention, R² is —O—(CH2)_{w}—R⁷ or —O—R⁷, and in yet another embodiment, R⁷ is a substituted or unsubstituted aryl or heteroaryl, wherein the number of substituents, if present, is in the range 1-3.

In another aspect, the invention provides compounds of Formula I wherein:
R¹ and R¹′ are independently a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R⁴ is hydrogen; and
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷′, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH2)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷.

In another embodiment of the invention, R² is —O—(CH₂)_{w}—R⁷ or —O—R⁷′. In yet another embodiment of the invention, R⁷ is a substituted or unsubstituted aryl or heteroaryl, wherein the number of substituents, if present, is in the range 1-3. In yet another embodiment of the invention, R⁷′ is a substituted or unsubstituted alkyl, cycloalkyl, cycloalkenyl, cycloalkylalkyl, cycloalkenylalkyl, saturated or unsaturated heterocyclyl, saturated or unsaturated heterocyclylalkyl group, aryl with amine substituent, or heteroaryl with amine substitution.

In another aspect, the invention provides compounds of Formula I wherein:
either R¹ or R¹′ is hydrogen;
R⁴ is not hydrogen; and
R² is —(CH₂)ₓ—R⁸′, —(CH₂)ₓ—NH(R⁹)—R⁸, —(CH₂)₂—O—R⁸′, —(CH₂)ₓ—S—R⁸, —O—(CH₂)_{w}—R⁸′, —NH(R⁹)—(CH₂)ₓ—R⁸, —S—(CH₂)ₓ—R⁸, —O—R⁸′, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸.

In another embodiment of the invention, R² is —O—(CH2)_{w}—R⁸ or —O—R⁸, and in yet another embodiment, R⁸ is a divalent substituted or unsubstituted aryl or heteroaryl group, each of which is attached to a substituted or unsubstituted heteroaryl group.

In another aspect, the invention provides compounds of Formula I wherein:
either R¹ or R¹′ is hydrogen;
R⁴ is hydrogen; and
R² is —(CH₂)ₓ—R⁸′, —(CH₂)ₓ—NH(R⁹)—R⁸, —(CH₂)_{w}—O—R⁸′, —(CH₂)ₓ—S—R⁸, —O—(CH₂)_{w}—R⁸′, —NH(R⁹)—(CH2)ₓ—R⁸′, —S—(CH₂)ₓ—R⁸, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸;.

In another aspect of this embodiment, R² is —O—(CH₂)_{w}—R⁸, and in yet another embodiment, R⁸ is a divalent substituted or unsubstituted aryl or heteroaryl group, each of which is attached to a substituted or unsubstituted heteroaryl group.

In another aspect, the invention provides compounds of Formula I wherein R³ and R³′ are independently hydrogen or a substituted or unsubstituted alkyl group, provided, however, that at least one of R³ and R³′ is hydrogen.

In another aspect, the invention provides compounds of Formula I wherein R⁴ and R⁵ are hydrogen.

In another aspect, the invention provides compounds of Formula I wherein A is selected from the group consisting of —C(O)— and —C(O)O—.

In another aspect, the invention provides compounds of Formula I wherein D is R⁶ or -alkyl-R⁶. In an embodiment of this aspect, R⁶ is a substituted or unsubstituted aryl, or heteroaryl group.

In another aspect, the invention provides compounds of Formula I wherein R⁹ is hydrogen.

In accordance with another aspect, the invention provides compounds of Formula II:

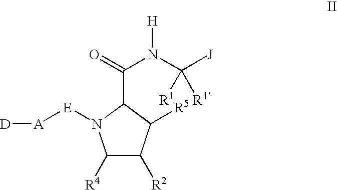

and stereoisomers, solvates, hydrates, tautomers, prodrugs, pharmaceutically acceptable salts, and mixtures thereof,

wherein J is:

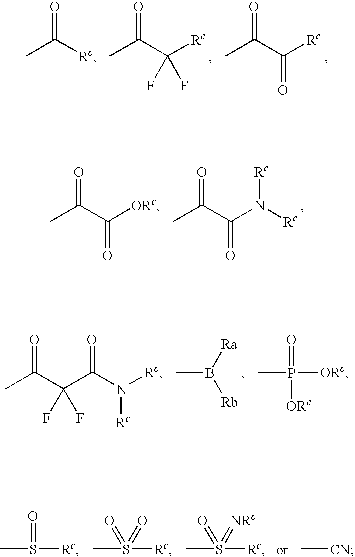

R^{c} at each occurrence is independently hydrogen, substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl; or
two R^{c} groups which are bound to the same nitrogen atom form together with said nitrogen atom a 5-7 membered monocyclic heterocyclic ring system;
A is a bond, —C(O)—, —C(O)O—, —C(O)NH—, —S(O)—, or —S(O)₂;
D is —R⁶, -alkyl-R⁶, -alkenyl-R⁶, -alkynyl-R⁶, —OR⁶, —NHR⁶, —N(R⁶)₂, or —C(O)R⁶;
E is a bond or

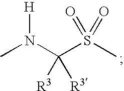

R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
R⁶ is a substituted or unsubstituted aryl, heteroaryl, cycloalkyl, heterocyclyl, cycloalkylidenyl, or heterocycloalkylidenyl group;
R⁷ at each occurrence is independently a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁹ is hydrogen or R⁷;
w is in the range 1-6; and
x is in the range 0-6.

In some embodiments, the invention provides compounds of Formula IIa:

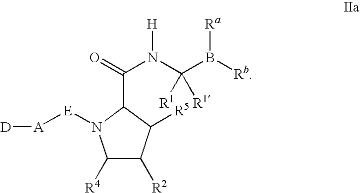

In some embodiments of compounds of Formula IIa, the invention provides compounds wherein R^{a} and R^{b} are independently hydroxyl, methoxy, ethoxy, n-propoxy, i-propoxy, or n-butoxy; or together are 1,2-dioxaethylene, 1,3-dioxapropylene, 1,3-dioxapropylene, 2,3-dimethyl-2,3-dioxabutane, or pinanedioxy.

In some embodiments, the invention provides compounds of Formula II wherein R¹ and R¹′ are independently hydrogen or a substituted or unsubstituted alkyl group.

In some embodiments, the invention provides compounds of Formula II wherein R² is —O—(CH₂)_{w}—R⁷. In some aspects of this embodiment, R⁷ is a substituted or unsubstituted aryl or heteroaryl, wherein the number of substituents, if present, is in the range 1-3.

In some embodiments, the invention provides compounds of Formula II wherein R⁴ and R⁵ are hydrogen.

In some embodiments, the invention provides compounds of Formula II wherein A is —C(O)— or —C(O)O—.

In some embodiments, the invention provides compounds of Formula II wherein D is R⁶ or -alkyl-R⁶. In some as aspects of this embodiment, the invention provides compounds of Formula II wherein R⁶ is a substituted or unsubstituted aryl, or heteroaryl group.

In some embodiments, the invention provides compounds of Formula II wherein R⁹ is hydrogen.

In some embodiments, the invention provides compounds of Formula II with structure of Formula IIb:

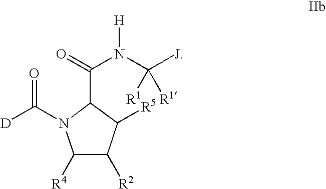

In some embodiments, the invention provides compounds of Formula II with structure of Formula IIc:

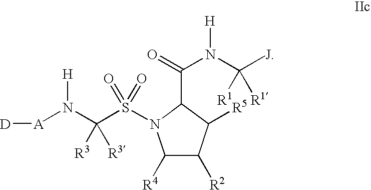

In some embodiments, the invention provides compounds of Formula II with structure of Formula IIc wherein R³′ is hydrogen.

In some embodiments, the invention provides compounds of Formula II with structure of Formula IId:

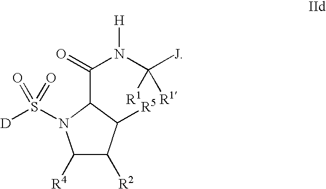

In accordance with another aspect, the invention provides compounds of Formula III:

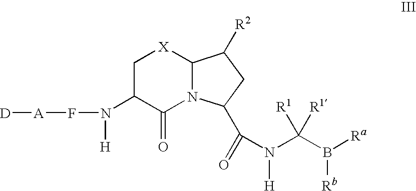

and stereoisomers, solvates, hydrates, tautomers, prodrugs, pharmaceutically acceptable salts, and mixtures thereof,

wherein:
X is CH₂ or S;
A is a bond, —C(O)—, —C(O)O—, —C(O)NH—, —S(O)—, or —S(O)₂—;
D is —R⁶, -alkyl-R⁶, -alkenyl-R⁶, -alkynyl-R⁶, —OR⁶, —NHR⁶, —N(R⁶)₂, or —C(O)R⁶;
F is a bond or

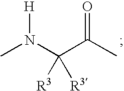

R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R²is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
R⁶ is a substituted or unsubstituted aryl, heteroaryl, cycloalkyl, heterocyclyl, cycloalkylidenyl, or heterocycloalkylidenyl group;
R⁷ is a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁹ is hydrogen or R⁷;
w is in the range 1-6; and
x is in the range 0-6.

In some embodiments of compounds of Formula III, the invention provides compounds wherein R^{a} and R^{b} are independently hydroxyl, methoxy, ethoxy, n-propoxy, i-propoxy, or n-butoxy; or together are 1,2-dioxaethylene, 1,3-dioxapropylene, 1,3-dioxapropylene, 2,3-dimethyl-2,3-dioxabutane, or pinanedioxy.

In some embodiments, the invention provides compounds of Formula III wherein R¹ and R¹′ are independently hydrogen or a substituted or unsubstituted alkyl group.

In some embodiments, the invention provides compounds of Formula III wherein R² is —O—(CH₂)_{w}—R⁷. In some aspects of this embodiment, R⁷ is a substituted or unsubstituted aryl or heteroaryl, wherein the number of substituents, if present, is in the range 1-3.

In some embodiments, the invention provides compounds of Formula III wherein A is —C(O)— or —C(O)O—.

In some embodiments, the invention provides compounds of Formula III wherein D is R⁶ or -alkyl-R⁶. In some aspects of this embodiment, the invention provides compounds of Formula III wherein R⁶ is a substituted or unsubstituted aryl, or heteroaryl group.

In some embodiments, the invention provides compounds of Formula III wherein R⁹ is hydrogen.

In some embodiments, the invention provides compounds of Formula III with structure of Formula IIIa:

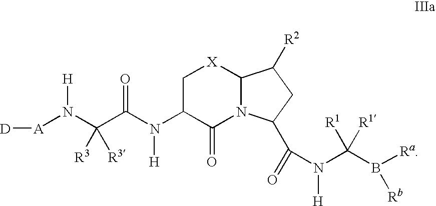

In some embodiments, the invention provides compounds of Formula III with structure of Formula IIIa wherein R³′ is hydrogen.

In some embodiments, the invention provides compounds of Formula III with structure of Formula IIIb:

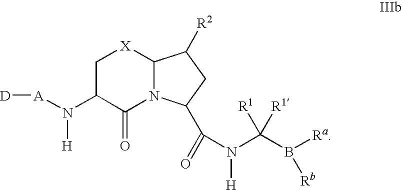

In accordance with another aspect, the invention provides compounds of Formula IV:

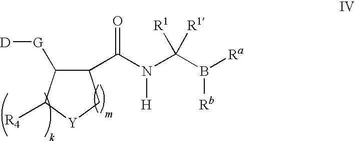

and stereoisomers, solvates, hydrates, tautomers, prodrugs, pharmaceutically acceptable salts, and mixtures thereof,

wherein:
D is —R⁶, -alkyl-R⁶, -alkenyl-R⁶, -alkynyl-R⁶, —OR⁶, —NHR⁶, —N(R⁶)₂, or —C(O)R⁶;
G is

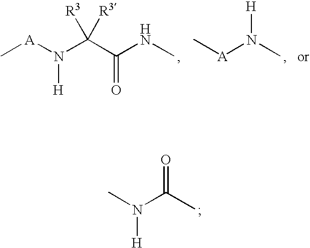

A is a bond, —C(O)—, —C(O)O—, —C(O)NH—,—S(O)—, —S(O)₂—;
Y is selected from the group consisting of O, S, CHR², and NR²′; such that
   when Y is CHR², then k is in the range 0-3, and m is in the range 0-3;
   when Y is NR²′, then k is in the range 1-3, and m is in the range 0-3; and
   when Y is O or Y is S, then k is in the range 1-3, and m is in the range 0-3;
R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R²′ is —(CH₂)ₓ—R⁷, —(CH₂)_{z}—NH(R⁹)—R⁷, —(CH₂)_{z}—O—R⁷, —(CH₂)_{z}—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
R⁶ is a substituted or unsubstituted aryl, heteroaryl, cycloalkyl, heterocyclyl, cycloalkylidenyl, or heterocycloalkylidenyl group;
R⁷ is a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁹ is hydrogen or R⁷;
w is in the range 1-6;
x is in the range 0-6; and
z is in the range 2-6.

In some embodiments of compounds of Formula IV, the invention provides compounds wherein R^{a} and R^{b} are independently hydroxyl, methoxy, ethoxy, n-propoxy, i-propoxy, or n-butoxy; or together are 1,2-dioxaethylene, 1,3-dioxapropylene, 1,3-dioxapropylene, 2,3-dimethyl-2,3-dioxabutane, or pinanedioxy.

In some embodiments, the invention provides compounds of Formula IV wherein R¹ and R¹′ are independently hydrogen or a substituted or unsubstituted alkyl group.

In some embodiments, the invention provides compounds of Formula IV with structure of Formula IVa:

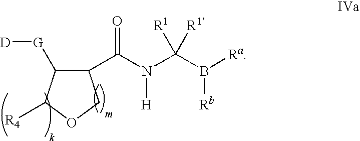

In some embodiments, the invention provides compounds of Formula IV with structure of Formula IVb:

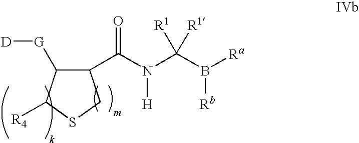

In some embodiments, the invention provides compounds of Formula IV with structure of Formula IVc:

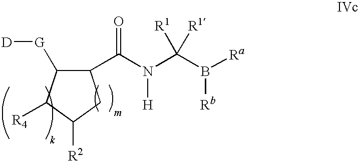

In some embodiments, the invention provides compounds of Formula IV with structure of Formula IVc wherein R² is —O—(CH₂)_{w}—R⁷, and wherein R⁷ is a substituted or unsubstituted aryl or heteroaryl, wherein the number of substituents, if present, is in the range 1-3.

In some embodiments, the invention provides compounds of Formula IV with structure of Formula IVd:

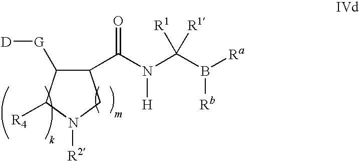

In some embodiments, the invention provides compounds of Formula IV with structure of Formula IVd wherein R² is —O—(CH₂)_{w}—R⁷, and wherein R⁷ is a substituted or unsubstituted aryl or heteroaryl, wherein the number of substituents, if present, is in the range 1-3.

In some embodiments, the invention provides compounds of Formula IV with structure of Formula IVe:

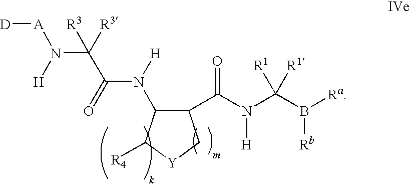

In some embodiments, the invention provides compounds of Formula IV with structure of Formula IVe wherein R³′ is hydrogen.

In some embodiments, the invention provides compounds of Formula IV with structure of Formula IVe wherein A is a bond, —C(O)—, or —C(O)O—.

In some embodiments, the invention provides compounds of Formula IV with structure of Formula IVf:

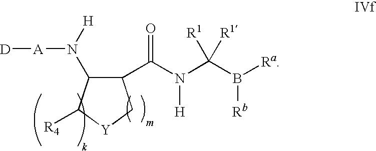

In some embodiments, the invention provides compounds of Formula IV with structure of Formula IVf wherein A is a bond, —C(O)—, or —C(O)O—.

In some embodiments, the invention provides compounds of Formula IV with structure of Formula IVg:

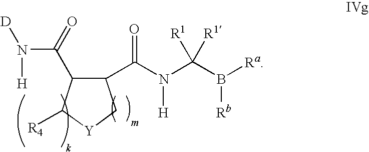

In some embodiments, the invention provides compounds of Formula IV wherein D is R⁶ or -alkyl-R⁶. In other aspects of this embodiment, R⁶ is a substituted or unsubstituted aryl, or heteroaryl group.

In some embodiments, the invention provides the compound of any of Formulae I-IV wherein:R⁷ is phenyl, quinolinyl, or quinazolinyl, each optionally substituted with 1-3 groups selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, quinazolinyl, and alkylamino substituted derivatives thereof.

In some embodiments, the invention provides the compound of Formula I wherein R⁷′ is phenyl-aminoheteroaryl, quinolinyl-aminoheteroaryl, quinazolinyl-aminoheteroaryl, each optionally substituted with one to three substituents selected from methoxy, phenyl, or both.

In some embodiments, the invention provides the compound of Formula I wherein R⁸ is phenyl, quinolinyl, or quinazolinyl, each optionally substituted with 1-3 groups selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, quinazolinyl, and alkylamino substituted derivatives thereof.

In some embodiments, the invention provides the compound of Formula I wherein R⁸′ is phenyl, quinolinyl, or quinazolinyl, each optionally substituted with 1-3 groups selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, quinazolinyl, and alkylamino substituted derivatives thereof.

In some embodiments, the invention provide the compound of any of Formulae I-IV wherein R⁷ is substituted with 1-3 groups selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

In some embodiments, the invention provides the compound of Formula I wherein R⁷′ is substituted with 1-3 groups selected from the group consisting of halogen, (C₁₋₁₀)alkyl, (C₁₋₁₀)alkoxy, (C₁₋₁₀)alkylamino, (C1-10) dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

In some embodiments, the invention provides the compound of Formula I wherein R⁸ is substituted with 1-3 groups selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

In some embodiments, the invention provides the compound of Formula I wherein R⁸′ is substituted with 1-3 groups selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

In accordance with another aspect, the present invention provides compounds of Formula V:

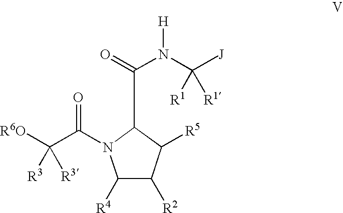

and stereoisomers, solvates, hydrates, tautomers, prodrugs, pharmaceutically acceptable salts, and mixtures thereof,

wherein:
J is

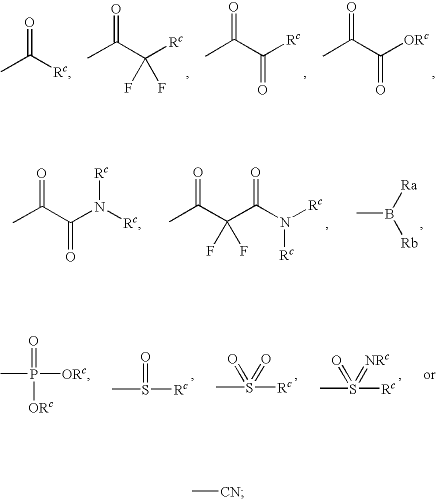

R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R^{c} at each occurrence is independently hydrogen, substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroararalyl; or
two R^{c} groups which are bound to the same nitrogen atom form together with said nitrogen atom a 5-7 membered monocylic heterocyclic ring system;
R¹ and R¹′ are independently hydrogen or a substituted or unsubstituted alkyl, alkenyl, alkynyl, cycloalkyl, cycloalkylalkyl, or arylalkyl group; or
   R¹ and R¹′ together with the carbon atom to which they are attached form a 3-7 membered substituted or unsubstituted carbocycle;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
provided that when R¹ and R¹′ are not hydrogen, then
   R² is —(CH₂)ₓ—R⁷, —(CH₂)ₓ—N(R⁹)—R⁷, —(CH₂)_{w}—O—R⁷, —(CH₂)ₓ—S—R⁷, —O—(CH₂)_{w}—R⁷, —N(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷; or R² and R⁴ together with the carbon atoms to which they are attached form a fused 3-6 member substituted or unsubstituted carbocyclyl or heterocyclyl group;
provided that when either R¹ or R¹′ is hydrogen, then
   R² is —(CH₂)ₓ—R⁸′, —(CH₂)ₓ—N(R⁹)—R⁸, —(CH₂)_{w}—O—R⁸′, —(CH₂)ₓ—S—R⁸, —O—(CH₂)_{w}—R⁸′, —N(R⁹)—(CH₂)ₓ—R⁸′, —S—(CH₂)ₓ—R⁸, —O—R⁸′, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸; or R² and R⁵ together with the carbon atoms to which they are attached form a fused 3-6 member substituted or unsubstituted carbocyclyl or heterocyclyl group;
R⁶ is hydrogen or a substituted or unsubstituted alkyl, alkenyl, alkynyl, aryl, heteroaryl, cycloalkyl, heterocyclyl, cycloalkylidenyl, or heterocycloalkylidenyl group;
R⁷ at each occurrence is independently a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁸ is a divalent substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R^{8′} is a divalent substituted or unsubstituted cycloalkyl, cycloalkenyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R⁹ is hydrogen or R⁷;
w is in the range 1-6; and
x is in the range 0-6.

In some embodiments of compounds of Formula V, J is

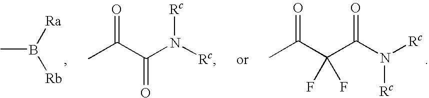

In accordance with another aspect, the present invention provides compounds of Formula Va:

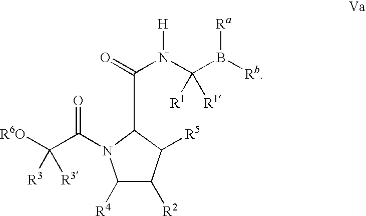

In some embodiments, the present invention provides compounds of Formulae V and Va wherein R^{a} and R^{b} are independently hydroxyl, methoxy, ethoxy, n-propoxy, i-propoxy, or n-butoxy; or together are 1,2-dioxaethylene, 1,3-dioxapropylene, 1,3-dioxapropylene, 2,3-dimethyl-2,3-dioxabutane, or pinanedioxy.

In further embodiments, the present invention provides compounds of Formula V wherein R¹ and R¹′ are independently hydrogen or a substituted or unsubstituted alkyl group.

In yet further embodiments, the present invention provides compounds of Formula V wherein R¹′ is hydrogen.

In still another embodiment, the present invention provides compounds of Formula V wherein R¹ is a C₁₋₈ substituted or unsubstituted alkyl, alkenyl, or cycloalkylalkyl group.

In certain aspects, the present invention provides compounds of Formula V wherein R² is —O—(CH₂)_{w}—R⁷ or —(CH₂)_{w}—O—R⁸′, and in accordance with this aspect there are further provided compounds of Formula V wherein R⁷ or R⁸′ is a substituted or unsubstituted aryl or heteroaryl, wherein the number of substituents, if present, is in the range 1-3.

In yet additional embodiments of this aspect of the present invention, compounds are provided wherein R² is 2-Pyrazol-1-yl-quinolin-4-olyl, 2-Pyrazol-1-yl-quinazolin-4-olyl, 2-(3-Methyl-pyrazol-1-yl)-quinolin-4-olyl, 2-(3-Methyl-pyrazol-1-yl)-quinazolin-4-olyl, 2-Pyrrolidin-1-yl-quinolin-4-olyl, 2-Pyrrolidin-1-yl-quinazolin-4-olyl, 2-(2-Methylamino-thiazol-4-yl)-quinolin-4-olyl, 2-(2-Methylamino-thiazol-4-yl)-quinazolin-4-olyl, 2-(2,2-Dimethylamino-thiazol-4-yl)-quinolin-4-olyl, 2-(2,2-Dimethylamino-thiazol-4-yl)-quinazolin-4-olyl, 2-(3,3-Difluoro-pyrrolidin-1-yl)-quinazolin-4-olyl, 2-(3,3-Difluoro-pyrrolidin-1-yl)-quinolin-4-olyl, 2-(2,3-Dimethyl-pyrrol-1-yl)-quinolin-4-olyl, 2-(2,3-Dimethyl-pyrrol-1-yl)-quinazolin-4-olyl, 2-Pyrrol-1-yl-quinolin-4-olyl, 2-Pyrrol-1-yl-quinazolin-4-olyl, 2-(3-Methyl-pyrrol-1-yl)-quinolin-4-olyl, 2-(3-Methyl-pyrrol-1-yl)-quinazolin-4-olyl, 2-(2-Isopropylamino-thiazol-4-yl)-quinolin-4-olyl, or 2-(2-Isopropylarnino-thiazol-4-yl)-quinazolin-4-olyl. These moieties are illustrated in the Chart below and are attached to the core pyrrolidine through the hydroxyl group.

CHART 1

| | |
|---|---|
| 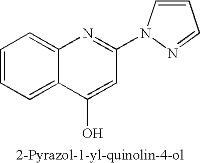 | 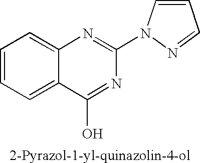 |
| 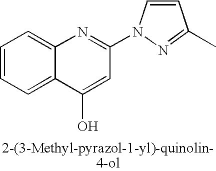 | 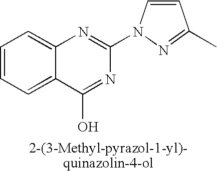 |
| 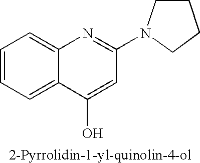 | 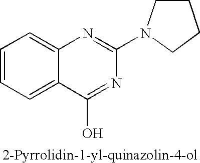 |
| 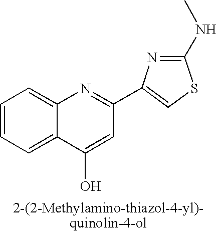 | 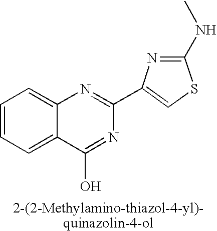 |
| 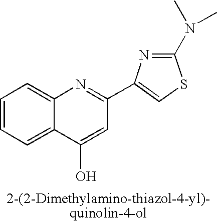 | 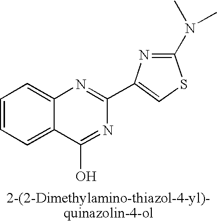 |
| 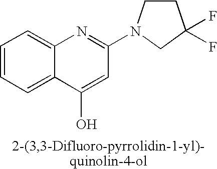 | 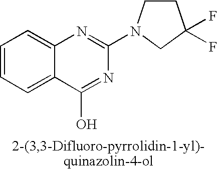 |
| 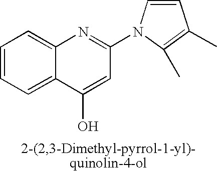 | 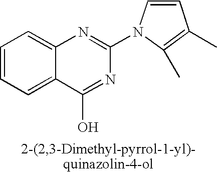 |
| 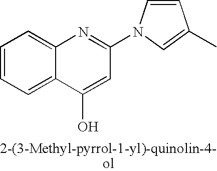 | 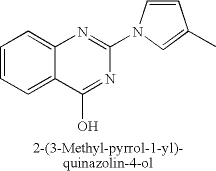 |
| 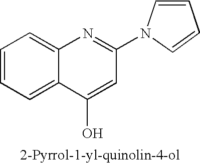 | 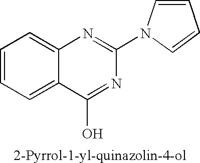 |
| 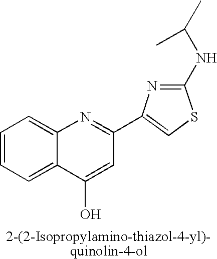 | 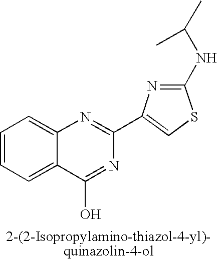 |

Further any of the embodiments which contemplate Formulae V or Va, the present invention further provides compounds wherein R⁸′ is attached to a monocyclic heteroaryl group.

Further any of the embodiments which embraces Formulae V or Va, the present invention further provides compounds

wherein:
R⁷ is phenyl, quinolinyl, or quinazolinyl, each optionally substituted with 1-3 substituents selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl, wherein each substituent is optionally substituted with 1 or 2 alkylamino groups.

Further any of the embodiments which embraces Formulae V or Va, the present invention further provides compounds

wherein:
R⁸ is phenyl, quinolinyl, or quinazolinyl, each optionally substituted with 1-3 substituents selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl, wherein each substituent is optionally substituted with 1 or 2 alkylamino groups.

Further any of the embodiments which embraces Formulae V or Va, the present invention further provides compounds

wherein:
R⁸′ is phenyl, quinolinyl, or quinazolinyl, each optionally substituted with 1-3 substituents selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, quinazolinyl, wherein each substituent is optionally substituted with 1 or 2 alkylamino groups.

Further any of the embodiments which embraces Formulae V or Va, the present invention further provides compounds

wherein:
R⁷ is substituted with 1-3 substituents selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

Further any of the embodiments which embraces Formulae V or Va, the present invention further provides compounds

wherein:
R⁸ is substituted with 1-3 substituents selected from the group consisting of halogen, (C₁₋₁₀)alkyl, (C₁₋₁₀)alkoxy, (C_{1-10-l )alkylamino, (C}₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

Further any of the embodiments which embraces Formulae V or Va, the present invention further provides compounds

wherein:
R⁸′ is substituted with 1-3 substituents selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

Further any of the embodiments which embraces Formulae V or Va, the present invention further provides compounds wherein R⁴ and R⁵ are hydrogen.

Further any of the embodiments which embraces Formulae V or Va, the present invention further provides compounds wherein R⁶ is hydrogen.

In accordance with another aspect, the present invention provides compounds of Formula V with structure of Formula Vb:

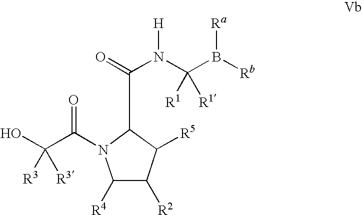

In certain embodiments, the present invention provides compounds of Formula V wherein R⁹ is hydrogen.

In certain embodiments, the present invention provides compounds of Formula V wherein R³′ is hydrogen.

In certain embodiments, the invention provides a method of inhibiting a hepatitis C viral protease comprising contacting said serine protease with a compound of Formulae I though V including all subdesignations such as IIa, IIIa, IVa and Va.

In certain embodiments, the invention provides a method for treating hepatitis C viral infection, comprising contacting said serine protease with a compound of Formulae V, Va, or Vb.

In a further embodiment, the invention provides a composition comprising a compound according to any of Formulae I through V including all subdesignations thereof and a pharmaceutically acceptable carrier.

In yet another embodiment, the invention provides a method for treating hepatitis C viral infection, comprising administering to a subject in need of such treatment an effective amount of the composition comprising a compound according to any of Formulae I through V including all subdesignations thereof, and a pharmaceutically acceptable carrier.

In another aspect, the invention provides a method for treating hepatitis C viral infection, comprising administering to a subject in need of such treatment an effective amount of a compound of any of Formulae I through V including all subdesignations thereof, in combination with another anti-viral agent. In certain embodiments of this aspect, the combined other anti-viral agent is another compound of Formulae I through V including all subdesignations thereof, as well as another, structurally different antiviral agent including but not limited to, Intron A, PEG-INTRON, Roferan A, Pegasys, Infergen A, Wellferon, ribavirin, ritonavir, nucleoside analogues, IRES inhibitors, NS5b inhibitors, E1 inhibitors, E2 inhibitors, IMPDH inhibitors, toll-like receptor agonists, NS5 polymerase inhibitors, or NTPase/helicase inhibitors and another NS3 protease inhibitor. Examples of other NS3 protease inhibitors which can be administered in combination with compounds of the present invention include, without limitation, VX950 (Lin C, Lin K, Luong Y, Rao B G, Wei Y Y, Brennan D L, Fulghum J R, Hsiao H M, Ma S, Maxwell J P, Cottrell K M, Pemi R B, Gates C A, Kwong A D, “In Vitro Resistance Studies of Hepatitis C Virus Serine Protease Inhibitors VX950 and BILN2061”, J. Biol. Chem., 2004, 279, 17508-17514), SCH503034 and ITMN191 as well as some of the antiviral agents mentioned above.

In a further embodiment, the invention provides a method for treating hepatitis C viral infection, comprising administering to a subject in need of such treatment effective amounts of a plurality of compounds of any of Formulae I-V and subdesignations thereof.

In yet a further embodiment, the invention provides a method for treating hepatitis C viral infection, comprising administering to a subject in need of such treatment effective amounts of a plurality of compounds of any of Formulae I-V and subdesignations thereof in combination with another anti-viral agent.

In yet a further embodiment, the invention provides a method for treating hepatitis C viral infection, comprising administering to a subject in need of such treatment an effective amount of a compound of any of Formulae I-V and subdesignations thereof in combination with another NS3 protease inhibitor.

In another aspect, the invention provides a method for treating hepatitis C viral infection, comprising administering to a subject in need of such treatment an effective amount of a compound of any of Formulae I through V including all subdesignations thereof in combination with an anti-proliferative agent. The term “anti-proliferative agent” as used herein denotes a compound which inhibits cellular proliferation. Cellular proliferation can occur, for example without limitation, during carcinogenesis, metastasis, and immune responses. In certain embodiments of this aspect, the anti-proliferative agent includes but is not limited to 5-fluorouracil, daunomycin, mitomycin, bleomycin, dexamethasone, methotrexate, cytarabine, or mercaptopurine.

In another aspect, the invention provides a method for treating hepatitis C viral infection, comprising administering to a subject in need of such treatment an effective amount of a compound of any of Formulae I through V including all subdesignations thereof, in combination with an immune modulator. The term “immune modulator” as used herein denotes a compound or composition comprising a plurality of compounds which changes any aspect of the functioning of the immune system. In this context, immune modulator includes without limitation anti-inflammatory agents and immune suppressants. In certain embodiments of this aspect, the immune modulator comprises a steroid, a non-steroidal anti-inflammatory, a COX2 inhibitor, an anti-TNF compound, an anti-IL-1 compound, methotrexate, leflunomide, cyclosporin, FK506, or a combination of any two or more thereof. In further embodiments of this aspect, the steroid comprises prednisone, prednisolone, or dexamethasone. In yet further embodiments of this aspect, the non-steroidal anti-inflammatory comprises ibuprofen, naproxen, diclofenac, or indomethacin. In still further embodiments of this aspect, the COX2 inhibitor comprises rofecoxib or celecoxib. In another embodiment of this aspect, the anti-TNF compound comprises enbrel, infliximab, or adalumimab. In yet another embodiment of this aspect, the anti-IL-1 compound comprises anakinra. Representative immune suppressants include without limitation cyclosporin and FK506.

In another aspect, the invention provides a method for treating hepatitis C viral infection, comprising administering to a subject in need of such treatment an NS3/4a inhibitor in combination with another anti-viral agent, wherein the NS3/4a inhibitor is a compound of Formulae I through V including all subdesignations thereof.

In yet another aspect, the invention provides a method for treating hepatitis C viral infection, comprising administering to a subject in need of such treatment an NS3/4a inhibitor in combination with an anti-proliferative agent, wherein the NS3/4a inhibitor is a compound of Formulae I through V including all subdesignations thereof.

In still another aspect, the invention provides a method for treating hepatitis C viral infection, comprising administering to a subject in need of such treatment an NS3/4a inhibitor in combination with immune modulators, wherein the NS3/4a inhibitor is a compound of Formulae I through V including all subdesignations thereof. In certain embodiments of this aspect, the present invention provides that the immune modulator includes but is not limited to a steroid, a non-steroidal anti-inflammatory, a COX2 inhibitor, an anti-TNF compound, an anti-IL-1 compound, methotrexate, leflunomide, cyclosporin, FK506, or a combination of any two or more thereof.

Further all embodiments of the present invention providing a method for treating hepatitis C viral infection comprising administering to a subject in need of such treatment an NS3/4a inhibitor or a compound of Formulae I through V including all subdesignations thereof, in combination with an immune modulator, the immune modulator is an anti-inflammatory or an immune suppressant.

Synthetic Methods

Without wishing to be bound by theory, the standard nomenclature of Schechter & Berger (Biochem. Biophys. Res. Comm., 1967, 27, 157-162) regarding the identification of residues in the polypeptide substrate of serine proteases will be employed herein unless other indicia of identification are specifically provided. Within the nomenclature of Schechter & Berger, the residues of the substrate, in the direction from the N-terminal toward the C-terminal, are labeled (Pi, . . . , P3, P2, P1, P1′, P2′, P3′, . . . , Pj), wherein cleavage is catalyzed between P1 and P1′. Within the context of this nomenclature, compounds of Formulae I, III, IV, and V can be considered as mimics of at least the tripeptide P3-Pro-P1, wherein P1 is

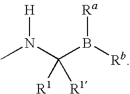

Similarly, in the context of compounds of the invention provided by Formula II, P1 finds a corresponding mimic in

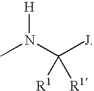

### Series A) P-1 Analogues: (S)-Configuration

Compounds of the invention with defined stereochemical configuration at the site which mimics the P1 site according to Schechter & Berger are available via the schemes following. As exemplified in Scheme A, P1 with structure

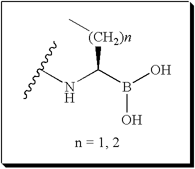

can be synthesized with absolute (S)-configuration by the incorporation of stereoselective protection of the terminal boron:

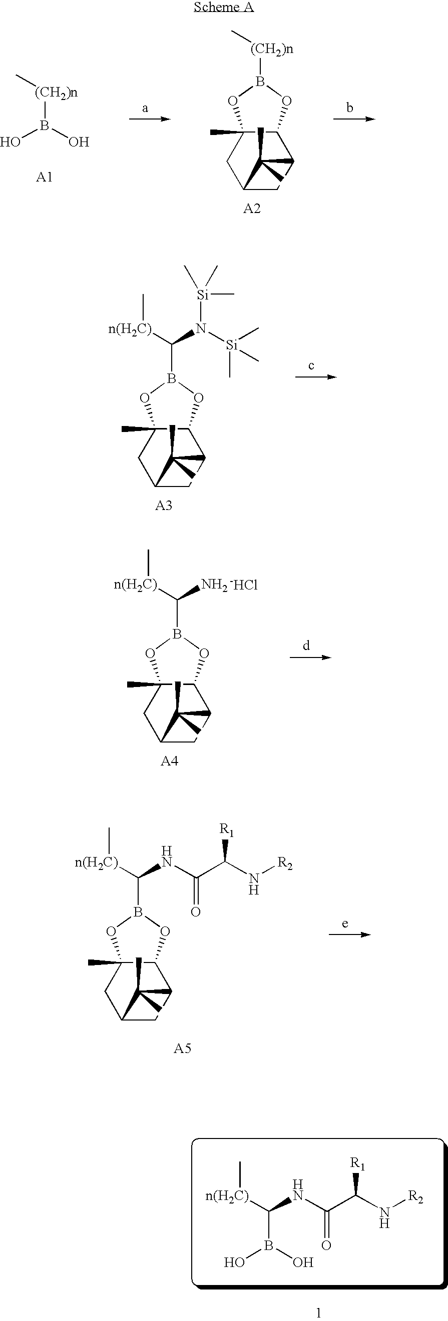

Step (a): Alkyldihydroxyborane A1 can react with (+)-pinanediol in Et₂O for 30 min to provide protected boronic acid A2. Step (b): Cmpd A2, in the presence of LiCHCl₂ at −100° C. then LiN(SiMe₃)₂ can form protected aminoalkylboronic acid A3. Step (c): In 4N HCl in dioxane at 0° C. A3 can be converted to protected aminoalkylboronic acid A4. Step (d): Peptide bond formation can be achieved with the reaction of A4 with EDC, HOBt, NMM, R²═PG-NH—CH(R)13 CO₂H, in DCM to provide Cmpd A5. Step (e): Deprotection of pinanediol provides the (S)-configuration of P1 analogue 1. Reference: Tetrahedron, 2003, 59, 579; Organometallics, 1984, 3, 1284.

### Series B) P-1 Analogues: (R)-Configuration

By symmetry with respect to Scheme 1, compounds of the invention with defined (R)-configuration at the P1 mimic site are available, as exemplified in Scheme B following wherein the P1 mimic site has structure

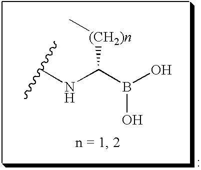

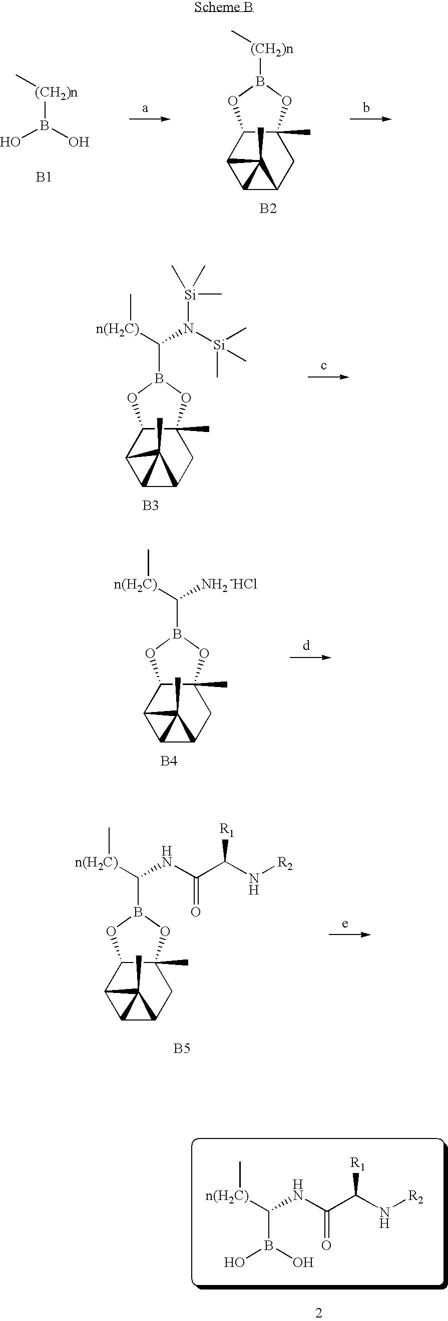

Step (a): Alkyldihydroxyborane B1 can react with (−)-pinanediol in Et₂O for 30 min to provide protected boronic acid B2. Step (b): Cmpd B2, in the presence of LiCHCl₂ at −100° C. then LiN(SiMe₃)₂ can form protected aminoalkylboronic acid B3. Step (c): In 4N HCl in dioxane at 0° C. B3 can be converted to protected aminoalkylboronic acid B4. Step (d): Peptide bond formation can be achieved with the reaction of B4 with EDC, HOBt, NMM, R₂═PG-NH—CH(R)—CO₂H, in DCM to provide Cmpd B5. Step (e): Deprotection of pinanediol provides the (S)-configuration of P1 analogue 2. Reference: Tetrahedron, 2003, 59, 579; Organometallics, 1984, 3, 1284.

### Series C) Cyclic P-1 Analogues

Compounds of the invention with structure of Formulae I-IV wherein R¹ and R¹′ form a 3-7 membered optionally substituted carbocycle are available via Scheme C:

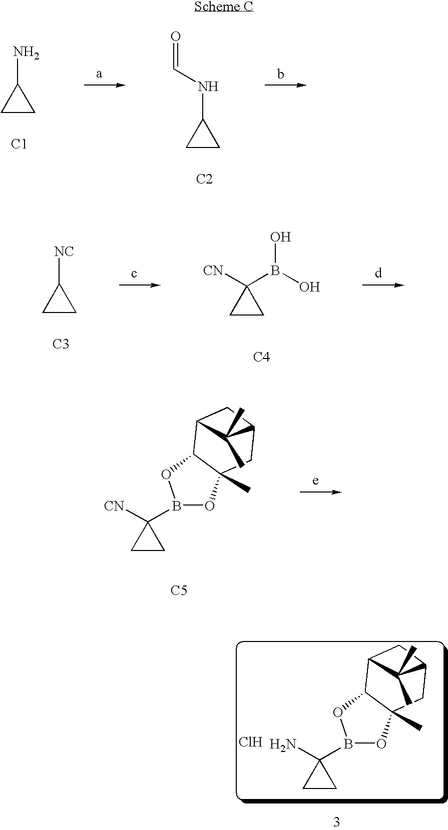

Step (a): Cycloalkylamine C1 can react with HCO₂H to provide cycloalkylformamide C2. Step (b): Upon reaction with Tos-Cl and Et3N, formamide C2 yields isocyanocycloalkyl C3. Step (c): Boration of C3 with n-BuLi, THF, −78° C., 30 min then triisopropylborate, −78° C. to RT, 14 h, can afford isocyanocycloalkylboronic acid C4. Step (d): Stereospecific protection of the boronic acid can be realized by reaction with (−)-pinanediol in Et₂O to afford cmpd C5. Step (e) Conversion to the amine salt in the presence of HCl and MeOH can afford cmpd 3. Reference: Org Lett., 2000, 20, 3095.

In addition to mimics of the P1 site, the present invention provides mimics of the P2 (i.e., Pro) site as a bicyclic proline. Representative synthetic schemes are given in Schemes D1-D2 below:

### Series D) Bicyclic P-2 Proline Mimics

Compounds of the invention with structure of

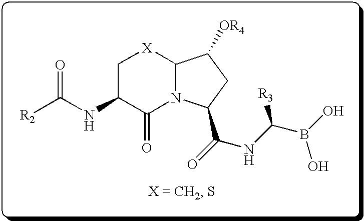

wherein X is CH₂ are available via Scheme D1:

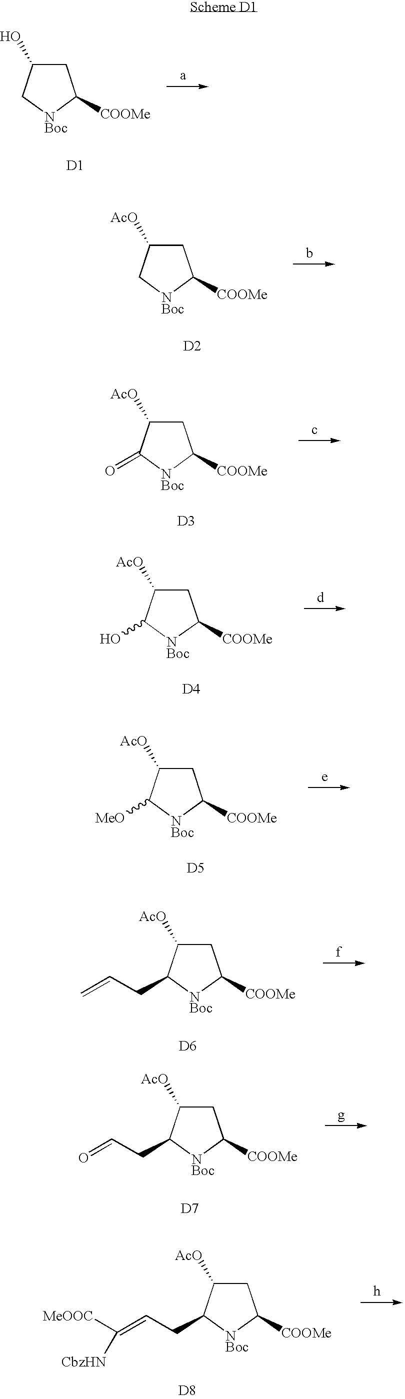

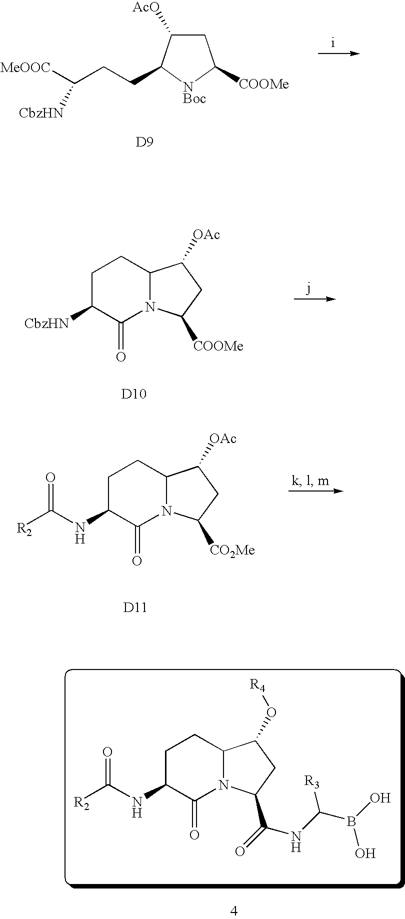

Step (a): Protected pyrrolidine methyl ester D1 can be acylated in reaction with Ac₂O in Py to afford Cmpd D2. Step (b): Oxidation of Cmpd D2 in the presence of KMnO₄, CuSO₄ and H₂O can afford pyrrolidinone D3. Step (c): Reduction of the ketone to the hydroxyl can be achieved with DIBAL-H in DCM to afford Cmpd D4. Step (d): Formation of the alkoxy cmpd D5 can be realized with H⁺ in MeOH. Step (e): Formation of alkene D6 can be achieved by reaction of D5 with BF₃OEt₂, TMSCH₂CH═CH₂ at −78° C. Step (f): Reaction of D6 with O₃; can provide aldehyde D7. Step (g): Reaction of aldehyde D7 with (MeO)₂P(O)CH(NHCbz)CO₂Me and DBU in DCM can provide Cmpd D8. Step (h): Reduction of D8 in the presence of Rh(I)(COD)-(s,s)-Et-DuPHOS, H₂ at 75 psi can provide Cmpd D9. Step (i): Ring closure of Cmpd D9 can be achieved with TFA in DCM, then TEA to provide Cmpd D10. Step (j): After hydrogenation j-i) of D10 [H₂, Pd on C in MeOH], peptide bond formation j-ii) can be realized with EDC, HOBt, R2═PG-NH—CH(R)—CO2H, NMM in DCM to afford Cmpd D11. Step (k): Reaction of D11 with a) NaOMe in MeOH followed by b) R⁴—OH, TPP, and DIAD in THF, 0° C. to RT in 2 days, can afford the R⁴—O— substituted reactant for Step L. Step L: Activation of the methyl ester [L-i) LiOH, THF—H₂O] followed by peptide bond formation [L-ii) EDC, HOBt, HCl—NH₂-boronic-AA-pinanediol] can afford the protected peptide reactant for Step m. Step (m): Deprotection of pinanediol can afford Cmpd 4. References: J. Org. Chem., 1995, 60, 5011; J. Org. Chem., 2000, 65, 10113; Tetrahedron Lett., 1988, 38, 329; J. Org. Chem., 1980, 55, 4817

Compounds of the invention with structure of

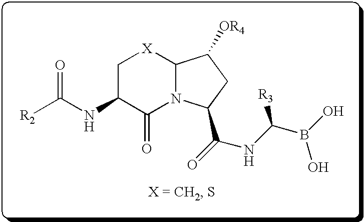

wherein X is S are available via Scheme D2:

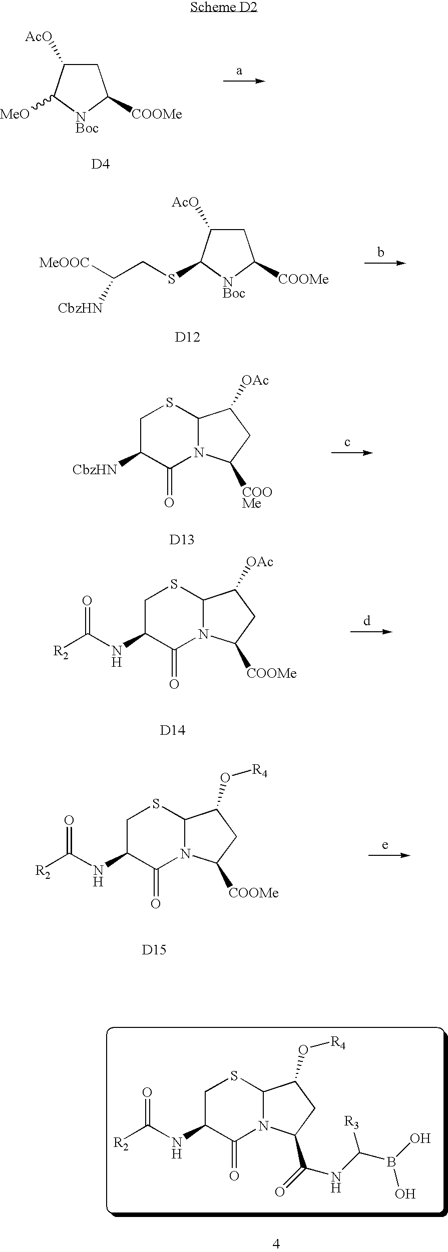

Step (a): Starting with Cmpd D4 (Scheme D1), reaction with BF₃—Et₂O and Cbz-Cys-Ome can provide Cmpd D12. Step (b): Pyrrolo[2,1-b]thiazineone D13 can result from ring closure of Cmpd D12 under the action TFA followed by TEA. Step (c): Hydrogenation of D13 [c-i): H2, Pd on C] followed by peptide bond formation [c-ii): EDC, HOBT, R²═PG-NH—CH(R)—CO2H, NMM in DCM] can afford Cmpd D14. Step (d): Deacylation [d-i): NaOMe in MeOH] followed by substitution [d-ii): R⁴—OH, TPP, DIAD in THF, 0° C. to RT, 2 days] can afford Cmpd D15. Step (e): Opening of the methyl ester [e-i): LiOH, THF—H₂O] followed by peptide bond formation [e-ii): EDC, HOBt, HCl—NH2-boronic-AA-pinanediol] and deprotection of pinanediol, can afford Cmpd 4. Leading References—Tetrahedron Lett., 1996, 46, 8395; J. Org. Chem., 1995, 60, 5011; J. Org. Chem., 2000, 65, 10113; Tetrahedron Lett., 1988, 38, 329; J. Org. Chem., 1980, 55, 4817.

### Series E) 4-Substituted Proline Analogues (5)

Compounds of the invention with structure of any of Formulae I-III with substitution at the 4-position of the pyrrolidine ring are available via Scheme E:

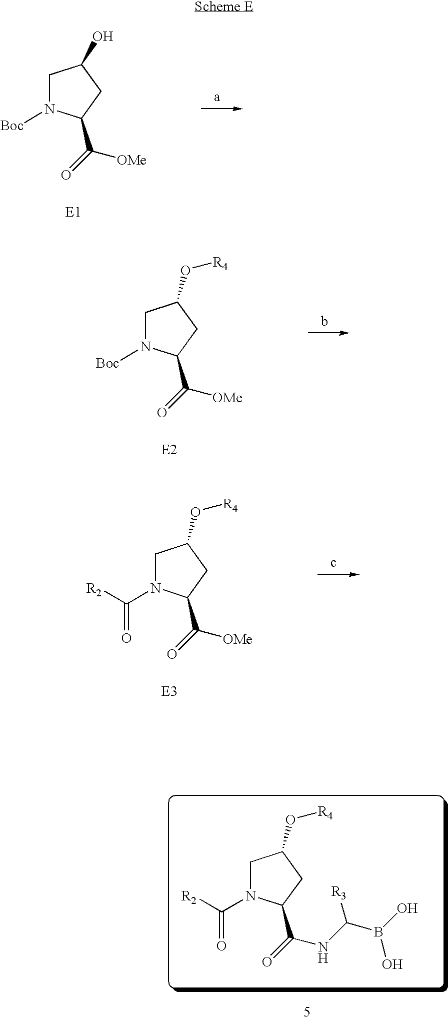

Step (a): Starting with protected hydroxypyrrolidine methyl ester E1, substitution of R⁴ can be realized under the reaction of R⁴—OH, TPP, and DIAD in THF, 0° C. to RT, 2 days, to afford Cmpd E2. Step (b): Formation of the amide can follow hydrogenation of E2 [b-i): H₂, Pd on C] and peptide bond formation [b-ii): EDC, HOBt, R²═PG-NH—CH(R)—CO2H, and NMM in DCM] to afford Cmpd E3. Step (c): Opening of the methyl ester of Cmpd E3 [c-i): LiOH and THF—H₂O] followed by peptide bond formation [c-ii): EDC, HOBt, HCl—NH2-boronic-AA-pinanediol] and deprotection of pinanediol can afford Cmpd 5. Leading References—Biopolymers, 2004, 76, 309; J. Med. Chem., 2004, 47, 6584; J. Med. Chem., 2004, 47, 123.; J. Chem. Soc. Perkin Trans I, 1994, 1411.

### Series F) Aminoproline Analogues (6): (“Trans” Configuration)

Compounds of the invention with structure of Formula IV with defined stereoconfiguration are available by synthetic schemes as provided by Scheme F:

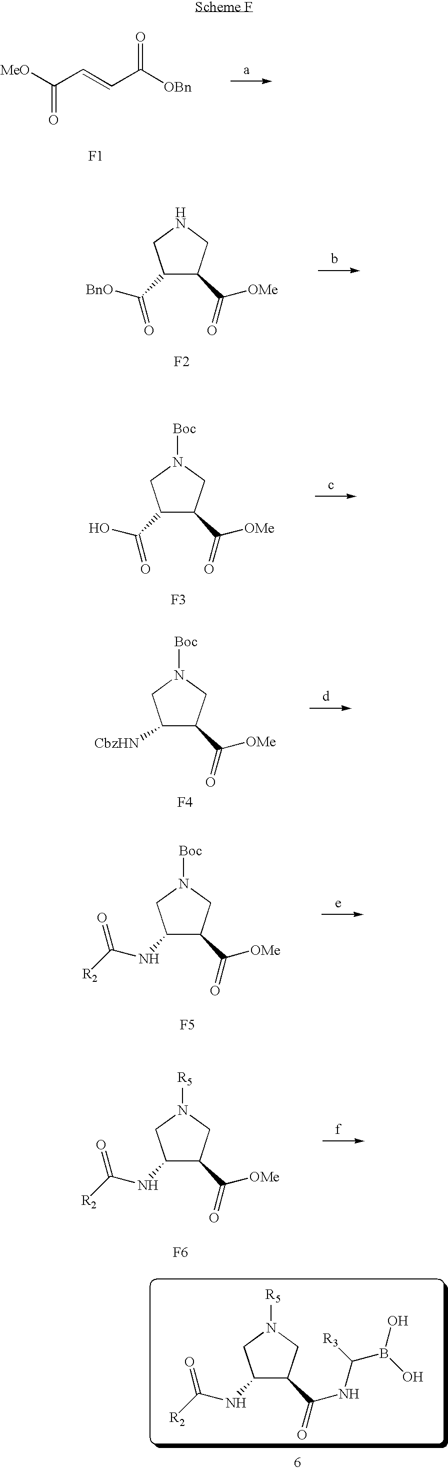

Step (a): Protected fumarate ester F1, in reaction with gly and CH₂O, in Tol at reflux, can provide protected pyrrolidine Cmpd F2. Step (b): Addition of Boc [b-i) (Boc)₂O in TEA] followed by hydrogenation [b-ii) H₂ on Pd(OH)₂] can afford Cmpd F3. Step (c): Formation of bis-protected Cmpd F4 can be realized by the reaction of F3 with DPPA, Et₃N, BnOH, and PhH at reflux. Step (d) Hydrogenation of Cmpd F4 [d-i): H₂/Pd on C] followed by peptide bond formation [d-ii): EDC, HOBt, R²═PG-NH—CH(R)—CO2H, and NMM in DCM] can afford Cmpd F5. Step (e): Removal of Boc [e-i): 4N HCl in dioxane] followed by substitution with R⁵ [e-ii): R⁵COCl, NMM in DCM] can afford Cmpd F6. Step (f): Opening of the methyl ester [f-i): LiOH in THF—H₂O] followed by peptide bond formation [f-ii): EDC, HOBt, HCl—NH2-boronic-AA-pinanediol] and deprotection of pinanediol can afford Cmpd 6. References—J. Chem Commun., 1985, 1566; DuPont Pharmaceuticals patent—WO 01/70673; Tetrahedron Lett., 1984, 25, 2557; J. Chem. Soc. Perkin Trans I, 1994, 1411.

### Series G) Aminoproline Analogues (7): (“Cis” Configuration)

Compounds of the invention with structure of Formula IV with defined stereoconfiguration are available by synthetic schemes as exemplified by Scheme G:

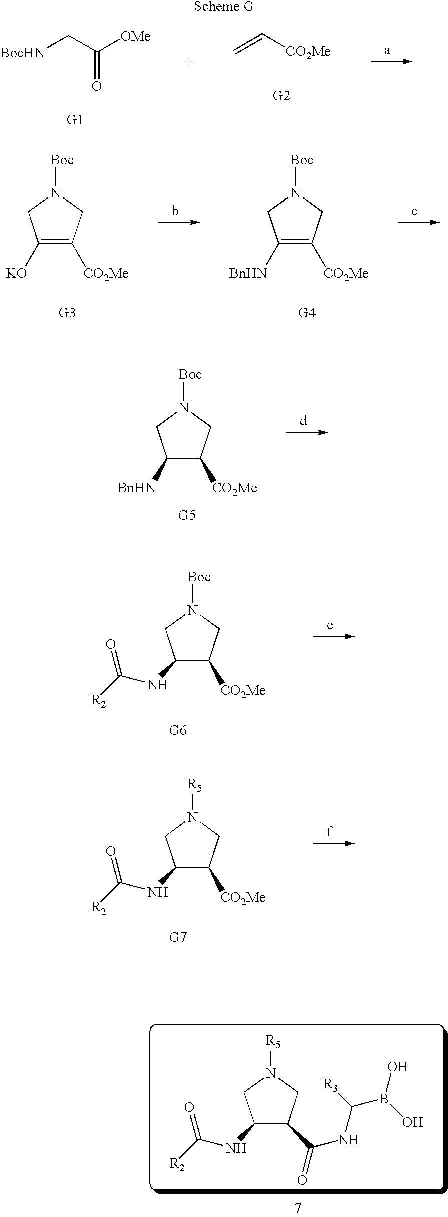

Step (a): Cycloaddition of protected amine methyl ester G1 and alkene methyl ester G2 in the presence of KOtBu in THF under reflux can provide dihydropyrrole G3. Step (b):Amination of G3 in the presence of BnNH₃OAc and HOAc in MeOH under reflux 1 h can provide dihydropyrrole G4. Step (c): Reduction to pyrrolidine G5 can occur in the presence of NaBH₄ and HOAc at 10° C. Step (d): Reduction of G5 [d-i) H2, Pd on C] followed by peptide bond formation [d-ii) EDC, HOBt, R2═PG-NH—CH(R)—CO2H, and NMM in DCM] can afford Cmpd G6. Step (e): Deprotection of Cmpd G6 [e-i) 4N HCl in dioxane] followed by substitution at nitrogen [e-ii) R⁵COCl and NMM in DCM] can afford Cmpd G7. Step (f): Cleavage of the methyl ester of G7 [f-i): LiOH, THF—H2O] followed by peptide bond formation [f-ii): EDC, HOBt, HCl—NH2-boronic-AA-pinanediol] and deprotection of pinanediol can afford Cmpd 7. References—Synthesis, 2001, 11, 1719; DuPont Pharmaceuticals patent—WO 01/70673; J. Med. Chem., 2001, 44, 1192; J. Org. Chem., 1960, 26, 1102; Chem Ber, 1956, 89, 1423; Pestic. Sci., 1997, 50, 329.

Series H) Hydroxycyclopentyl-(cis)-1,2-dicarboxylic acids (8) and hydroxycyclopentyl-(cis)-β-amino acid (9)

Compounds of the invention with structure of Formula IV with defined stereoconfiguration are available by synthetic schemes as exemplified by Scheme H:

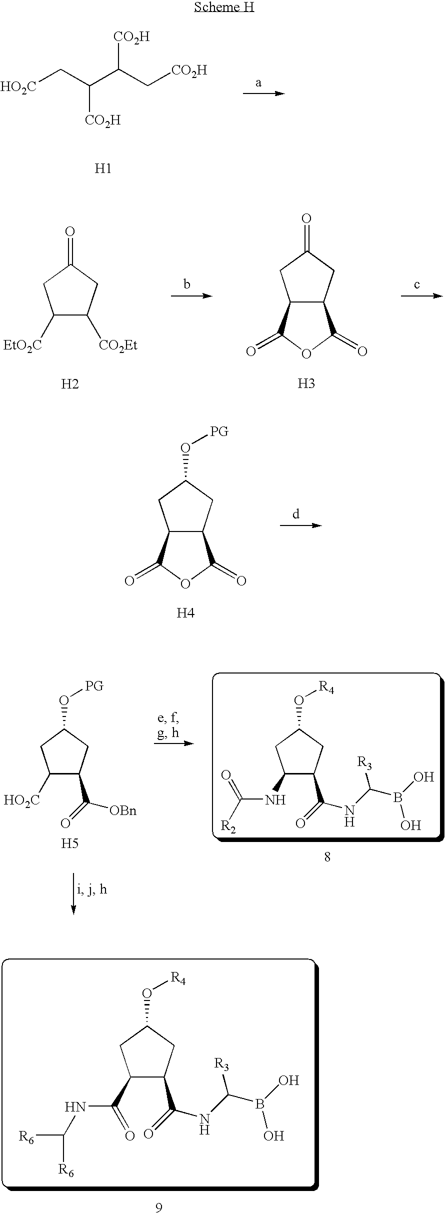

Step (a): Tetracarboxylic acid H1 can cyclize in reaction with [a-i): EtOH, H₂SO₄], [a-ii): NaOMe, MeOH], [a-iii): HCl, H₂O], and [a-iv): EtOH, H₂SO₄] to form cyclopentyl ketone H2. Step (b): Formation of the anhydride H3 can proceed with (EtCO)₂O at 135° C. Step (c): Reduction of the ketone with NaCNBH₃, followed by adduction of protecting group PG can afford Cmpd H4. Step (d): Formation of the vicinal diacid H5 can proceed under reaction with BnOH and cat. quinine base. Starting with reagent H5, the synthetic path bifurcates, leading to Cmpd 8 via steps e-h, and to Cmpd 9 via step i-j, and h. Step (e): [e-i) EtCOCl, TEA, NaN₃, Bn-OH] [e-ii) H2, Pd on C]; Step (f): EDC, HOBt, R²═PG-NH—CH(R)—CO₂H, NMM, DCM; Step (g): deprotect PG, then R⁴—OH, TPP, DIAD, THF 0° C. to RT, 2 days; Step (h) [h-i): NaOH, THF—H2O, reflux] [h-ii): EDC, HOBt, HCl—NH2-boronic-AA-pinanediol] [h-iii): deprotection pinanediol] to afford Cmpd 8. Step (i) deprotect PG, then R⁴—OH, TPP, DIAD, THF 0° C. to rt, 2 days. Step (j) EDC, HOBt, (R6)2-NH2, NMM, DCM; followed by Step (h) to afford Cmpd 9. References—For compound 8—Tetrahedron, 2002, 58, 8629; Bioorg. Med. Chem. Lett., 2003, 13, 433; Tetrahedron Asymmetry, 2003, 14, 3455; Chem. Rev., 2001, 101, 2181.; For compound 9—Chem. Rev., 2001, 101, 2181; J. Med. Chem., 1993, 36, 699; Bioorg. Med. Chem. Lett., 2003, 13, 4293; J. Med Chem., 2003, 46, 1165; J. Med. Chem., 2000, 43, 1705.

Series I) Hydroxycyclohexyl-(cis)-1,2-dicarboxylic acids and hydroxycyclohexyl-(cis)-β-amino acid

Compounds of the invention with structure of Formula IV with defined stereoconfiguration are available by synthetic schemes as exemplified by Scheme I:

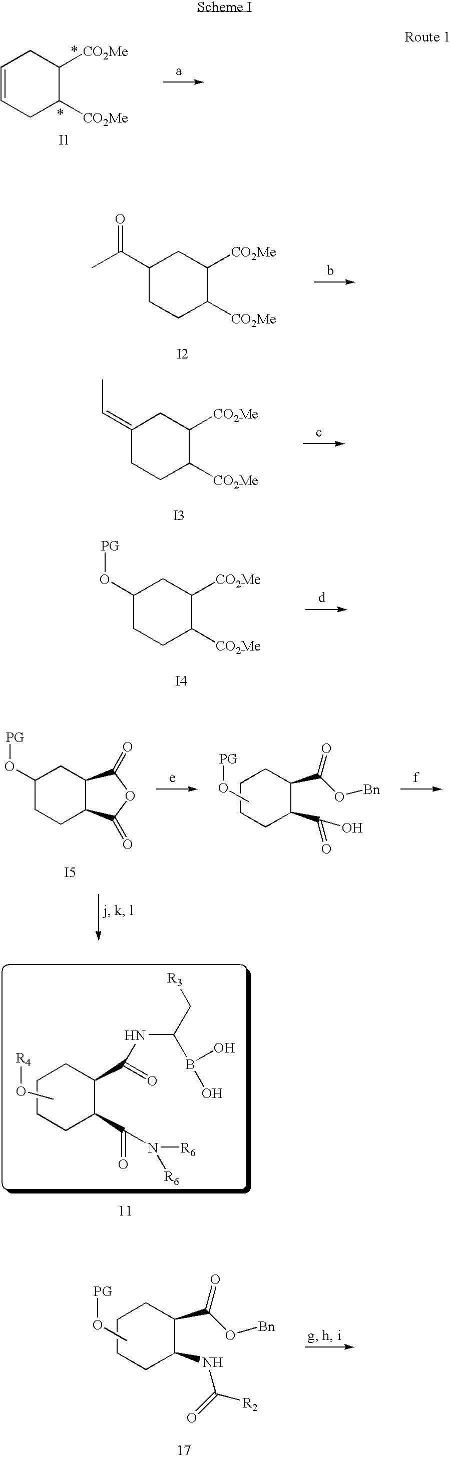

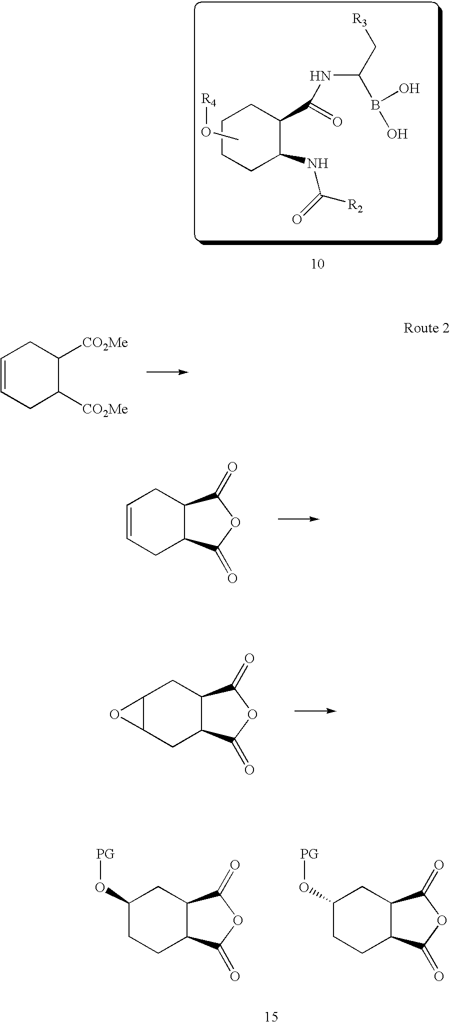

Step (a): Ac₂O, SnCl₄; then H⁺, MeOH. Step (b) p-TsNHNH₂, EtOH, reflux; then catecholborane, 0° C., NaOAc. Step (c) O3; then DIBAL-H, then protection with protecting group PG. Step (d): (EtCO)2O, 135° C. Step (e): BnOH, quinine, −15° C. Step (i): Step f-i) EtCOCl, TEA, NaN3, Bn-OH; Step f-ii) H₂, Pd on C; Step f-iii) EDC, HOBt, R²═PG-NH—CH(R)—CO2H, NMM, DCM. Step (g): deprotect PG, then R⁴—OH, TPP, DIAD, THF 0° C. to rt, 2 days. Step (h): Step h-i): NaOH, THF—H₂O, reflux; Step h-ii) EDC, HOBt, HCl—NH2-boronic-AA-pinanediol. Step (i): deprotection pinanediol. Step (j): deprotect PG, then R⁴—OH, TPP, DIAD, THF 0° C. to rt, 2 days. Step (k): Step k-i) NaOMe, MeOH; Step k-ii) EDC, HOBt, (R⁶)₂—NH2, NMM, DCM. Step (j): Step j-i) LiOH, THF—H2O, followed by EDC, HOBt, HCl—NH2-boronic-AA-pinanediol; Step j-ii) deprotection pinanediol. References—Tetrahedron, 1984, 40, 4677; J. Med. Chem., 1996, 61, 5557; Chem. Rev., 2001, 101, 2181; Bioorg. Med. Chem. Lett., 1998, 8, 1249; Org. Biomol. Chem., 2004, 2, 1105; J. Med. Chem., 1993, 36, 699; Tetrahedron, 2001, 57, 3175; Tetrahedron Asymmetry, 2004, 15, 3545; Bioorg. Med. Chem. Lett., 2003, 13, 4293; J. Med. Chem., 1997, 40, 3119.

Series J) Hydroxycyclohexyl-(trans)-1,2-dicarboxylic acids (12) and hydroxycyclohexyl-(trans)-β-amino acid (13), hydroxypentyl-(trans)-b-amino acids (14), and hydroxycyclopentyl-(trans)-1,2-dicarboxylic acids (15)

Compounds of the invention with structure of Formula IV with defined stereoconfiguration are available by synthetic schemes as exemplified by Scheme J:

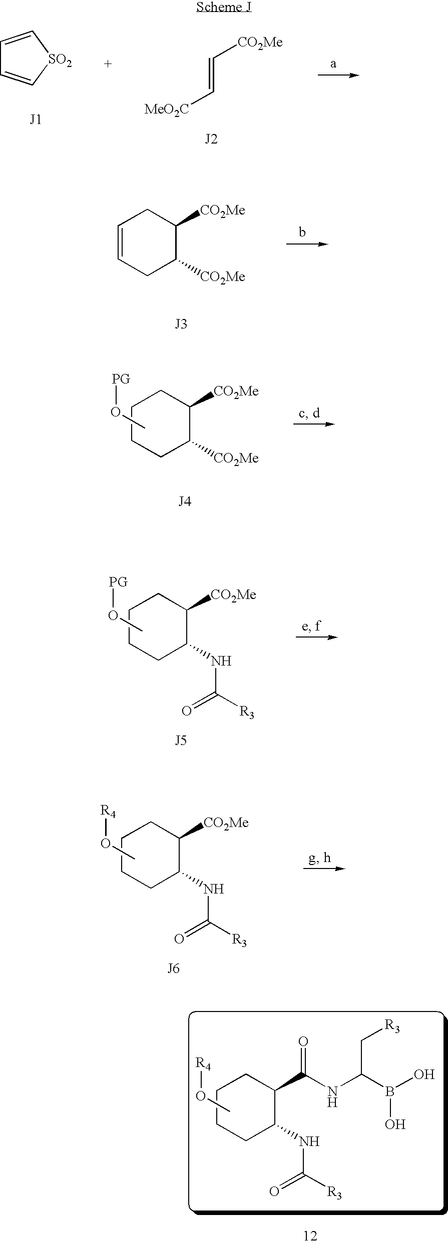

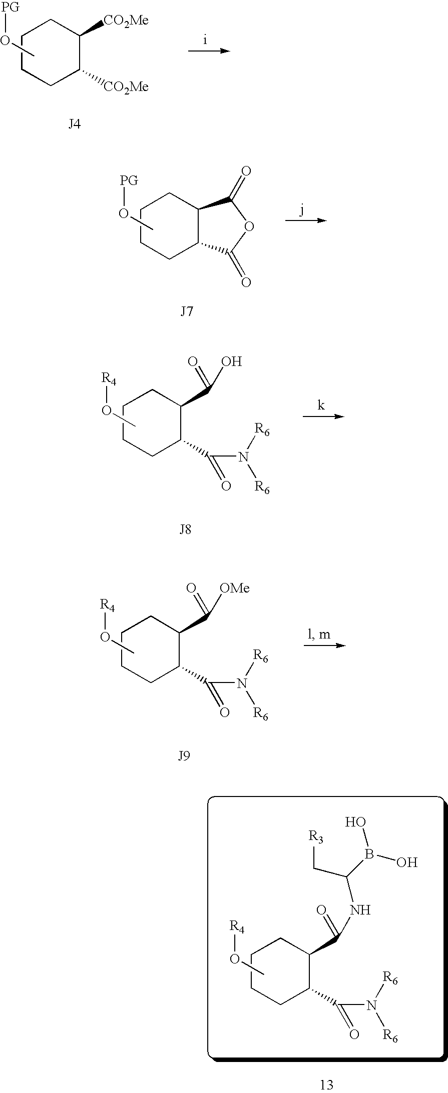

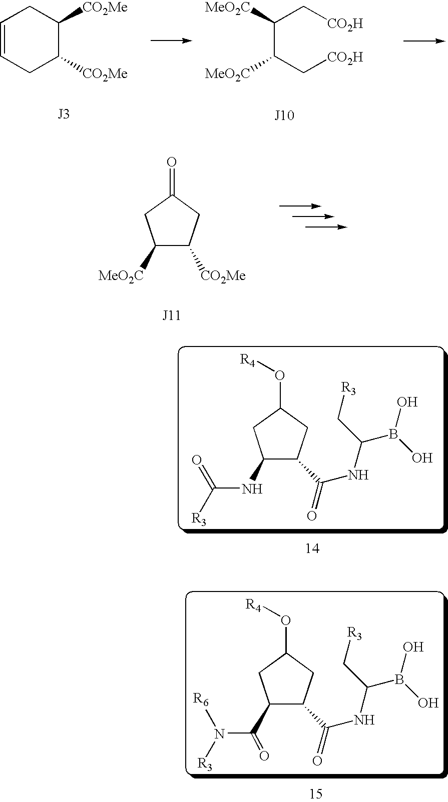

Step (a): Reagent J2 and J3, neat, in sealed tube, 100° C. Step (b): mCPBA or Step b-i) Ac₂O, SnCl₄; then H⁺, MeOH; Step b-ii) p-TsNHNH₂, EtOH, reflux; then catecholborane, 0° C., NaOAc; c) O₃; then DIBAL-H, then protect. Step (c): Step c-i) EtCOCl, TEA, NaN₃, Bn-OH; Step c-ii) H₂, Pd on C. Step (d): EDC, HOBt, R²═PG-NH—CH(R)—CO₂H, NMM, DCM. Step (e) deprotect PG, then R⁴—OH, TPP, DIAD, THF 0° C. to RT, 2 days;

Step (f): Step f-i) NaOMe, MeOH; Step f-ii) EDC, HOBt, (R⁶)₂—NH2, NMM, DCM; Step (g): Step g-i) LiOH, THF—H2O, followed by EDC, HOBt, HCl—NH2-boronic-AA-pinanediol; Step g-ii) deprotection pinanediol; Step (i)deprotect PG, then R⁴—OH, TPP, DIAD, THF 0° C. to RT, 2 days; Step (j) Step j-i) NaOMe, MeOH; Step j-ii) EDC, HOBt, (R6)2-NH2, NMM, DCM; Step (k): Step k-i) LiOH, THF—H2O, followed by EDC, HOBt, HCl—NH2-boronic-AA-pinanediol; Step k-ii) deprotection pinanediol. Note-14 and 15 can be accessed via route described in Series H (Scheme 9). References—J. Med. Chem., 1997, 40, 3119; Tetrahedron, 2002, 58, 8629; Bioorg. Med. Chem. Lett., 2003, 13, 433; Tetrahedron Asymmetry, 2003, 14, 3455; Chem. Rev., 2001, 101, 2181.; Chem. Rev., 2001, 101, 2181; J. Med. Chem., 1993, 36, 699; Bioorg. Med. Chem. Lett., 2003, 13, 4293.

### Series K) 4,5-Di-substituted Proline Analogues (16)

Compounds of the invention with structure of Formula IV with defined stereoconfiguration are available by synthetic schemes as exemplified by Scheme K:

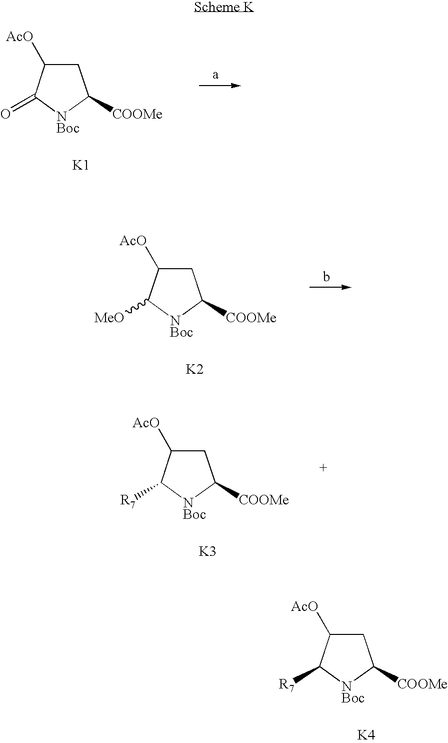

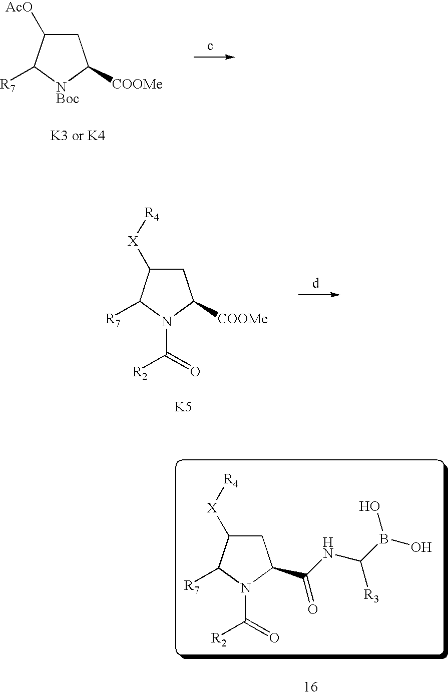

Step (a): DIBAL-H, THF, then H+, MeOH. Step (b): R—MgCl, CuBr-Me₂S, BF₃—OEt₂, (e) a) 4N HCl in dioxane; b) EDC, HOBt, R²═PG-NH—CH(R)—CO2H, NMM, DCM; (d) a) deprotect PG, then R⁴—OH, TPP, DIAD, THF 0° C. to RT, 2 days; b) LiOH, TFH-H20; c) EDC, HOBt, HCl—NH2-boronic-AA-pinanediol; d) deprotection pinanediol. References—Tetrahedron, 2001, 57, 6455; J. Org. Chem., 1995, 60, 5011; J. Org. Chem., 2000, 65,10113; Tetrahedron Lett., 1988, 38, 329; J. Org. Chem., 1980, 55, 4817

### Series L) Aminopiperidine Analogues (17)

Compounds of the invention with structure of Formula IV with defined stereoconfiguration are available by synthetic schemes as exemplified by Scheme L:

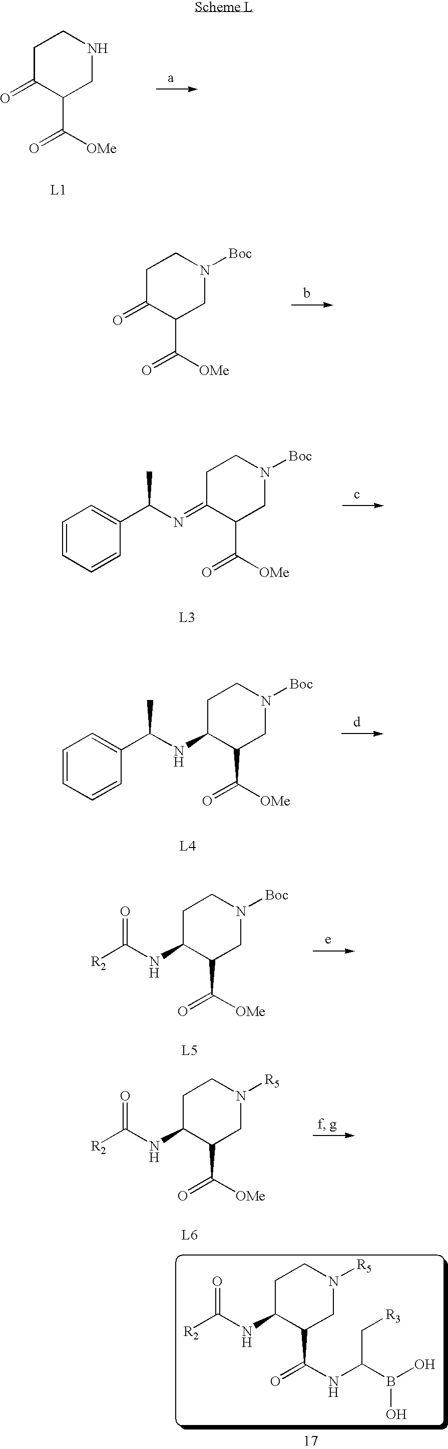

Step (a): (Boc)₂O, TEA. Step (b): Yb(OTf)₃, (R)-α-MeBnNH₂, reflux, dean-stark trap. Step (c): NaHB(OAc)₃, HOAc, 0° C. Step (d): Step d-i) H2, Pd on C; Step d-ii) EDC, HOBt, R²═PG-NH—CH(R)—CO2H, NMM, DCM. Step (e): RCOCl, NMM, DCM. Step (f): Step f-i) LiOH, TFH—H2O; Step f-iib) EDC, HOBt, HCl—NH2-boronic-AA-pinanediol; Step f-iii) deprotection pinanediol. References—DuPont Pharmaceuticals patent WO 01/70673;

### Methods/Uses

In some embodiments, the invention provides a method of inhibiting a hepatitis C viral protease comprising contacting said serine protease with a compound of any of the foregoing Formulae I-V, including subdesignations thereof.

In some embodiments, the invention provides a method for treating hepatitis C viral infection, comprising administering to a subject in need of such treatment an effective amount of a compound of any of foregoing Formulae I-V, including subdesignations thereof.

In another embodiment, the invention provides a composition comprising a compound according to any of the foregoing Formulae I-V, including subdesignations thereof and a pharmaceutically acceptable carrier.

In yet another embodiment, the invention provides a method for treating hepatitis C viral infection, comprising administering to a subject in need of such treatment an effective amount of the composition comprising a pharmaceutically acceptable carrier and a compound according to any of Formulae I-V, including subdesignations thereof, or a combination of one or more compounds according to any of Formulae I-V including subdesignations thereof and another structurally different compound including but not limited to an antiviral agent, an anti-proliferative agent, an immune modulator, and an NS3 protease inhibitor.

Compounds of the invention include mixtures of stereoisomers such as mixtures of diastereomers and/or enantiomers. In some embodiments, the compound, e.g. of Formulae I, II, III, IV or V or subdesignations thereof, is 90 weight percent (wt %) or greater of a single diastereomer of enantiomer. In other embodiments, the compound is 92, 94, 96, 98 or even 99 wt % or more of a single diastereomer or single enantiomer.

A variety of uses of the invention compounds are possible along the lines of the various methods of the treating an individual such as a mammal described above. Exemplary uses of the invention methods include, without limitation, use of a compound of the invention for the manufacture of a medicament for treating a condition that is regulated or normalized via inhibition of the HCV NS3 serine protease.

Biochemical Methods

Fluorescence resonance energy transfer (FRET; see e.g., Heim et al., (1996) Curr. Biol. 6:178-182; Mitra et al., (1996) Gene 173:13-17; and Selvin et al., (1995) Meth. Enzymol. 246:300-345) is an exquisitely sensitive method for detecting energy transfer between two fluorophoric probes. As known in the art, such probes are given the designations “donor” and “acceptor” depending on the relative positions of the maxima in the absorption and emission spectra characterizing the probes. If the emission spectrum of the acceptor overlaps the absorption spectrum of the donor, energy transfer can occur. Because of the known and highly non-linear relationship of energy transfer and distance between fluorophores, approximated by an inverse sixth power dependence on distance, FRET measurements correlate with distance. For example, when the probes are in proximity, such as when the probes are attached to the N— and C-termini of a peptide substrate, and the sample is illuminated in a spectrofluorometer, resonance energy can be transferred from one excited probe to the other resulting in observable signal. Upon scission of the peptide linking the probes, the average distance between probes increases such that energy transfer between donor and accept probe is not observed. As a result, the degree of hydrolysis of the peptide substrate, and the level of activity of the protease catalyzing hydrolysis of the peptide substrate, can be quantitated. Accordingly, using methods known in the arts of chemical and biochemical kinetics and equilibria, the effect of inhibitor on protease activity can be quantitated.

Compositions and Combination Treatments

A. Compositions. Another aspect of the invention provides compositions of the compounds of the invention, alone or in combination with another NS3 protease inhibitor or another type of antiviral agent and/or another type of therapeutic agent. Compositions containing a compound of the invention may be prepared by conventional techniques, e.g. as described in Remington: The Science and Practice of Pharmacy, 19th Ed., 1995. The compositions may appear in conventional forms, for example capsules, tablets, aerosols, solutions, suspensions or topical applications.

Typical compositions include a compound of the invention which inhibits the enzymatic activity of the HCV NS3 protease, and a pharmaceutically acceptable excipient which may be a carrier or a diluent. Compounds of the invention include compounds of Formula I, II, III, IV, or V or subdesignations thereof, and combinations of compounds thereof. As set forth herein, compounds of the invention include stereoisomers, tautomers, solvates, prodrugs, pharmaceutically acceptable salts and mixtures thereof. The compound may be diluted by a carrier, or enclosed within a carrier which can be in the form of a capsule, sachet, paper or other container. In making the compositions, conventional techniques for the preparation of compositions in the pharmaceutical arts may be used.

For example, the active compound will usually be mixed with a carrier, or diluted by a carrier, or enclosed within a carrier which may be in the form of an ampoule, capsule, sachet, paper, or other container. When the active compound is mixed with a carrier, or when the carrier serves as a diluent, it may be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration may be any route which effectively transports the active compound of the invention which inhibits the enzymatic activity of the HCV NS3 protease to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

Injectable dosage forms generally include aqueous suspensions or oil suspensions which may be prepared using a suitable dispersant or wetting agent and a suspending agent Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the formulation may also be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers.

The formulations of the invention may be designed to provide quick, sustained, or delayed release of the active ingredient after administration to the patient by employing procedures well known in the art. Thus, the formulations may also be formulated for controlled release or for slow release.

Compositions contemplated by the present invention may comprise, for example, micelles or liposomes, or some other encapsulated form, or may be administered in an extended release form to provide a prolonged storage and/or delivery effect. Therefore, the formulations may be compressed into pellets or cylinders and implanted intramuscularly or subcutaneously as depot injections or as implants such as stents. Such implants may employ known inert materials such as silicones and biodegradable polymers, e.g., polylactide-polyglycolide. Examples of other biodegradable polymers include poly(orthoesters) and poly(anhydrides).

For nasal administration, the preparation may contain a compound of the invention which inhibits the enzymatic activity of the HCV NS3 protease, dissolved or suspended in a liquid carrier, preferably an aqueous carrier, for aerosol application. The carrier may contain additives such as solubilizing agents, e.g., propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that may be prepared by conventional tabletting techniques may contain:

Core:

| | |
|---|---|
| Active compound (as free compound or salt thereof) | 250 mg |
| Colloidal silicon dioxide (Aerosil) ® | 1.5 mg |
| Cellulose, microcryst. (Avicel) ® | 70 mg |
| Modified cellulose gum (Ac-Di-Sol) ® | 7.5 mg |
| Magnesium stearate | Ad. |

Coating:

| | |
|---|---|
| HPMC approx. | 9 mg |
| *Mywacett 9-40 T approx. | 0.9 mg |

*Acylated monoglyceride used as plasticizer for film coating.

The compounds of the invention may be administered to a mammal, especially a human in need of such treatment, prevention, elimination, alleviation or amelioration of the various diseases as mentioned above, e.g., HCV infection. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg. A typical dosage is about 10 mg to about 1000 mg per day. The exact dosage will depend upon the activity of the compound, mode of administration, on the therapy desired, form in which administered, the subject to be treated and the body weight of the subject to be treated, and the preference and experience of the physician or veterinarian in charge. HCV NS3 protease inhibitor activity of the compounds of the invention may be determined by use of an in vitro assay system which measures the potentiation of inhibition of the HCV NS3 protease. Inhibition constants (i.e., Kᵢ or IC₅₀ values as known in the art) for the HCV NS3 protease inhibitors of the invention may be determined by the method described in the Examples.

Generally, the compounds of the invention are dispensed in unit dosage form comprising from about 1 mg to about 1000 mg of active ingredient together with a pharmaceutically acceptable carrier per unit dosage.

Usually, dosage forms suitable for oral, nasal, pulmonal or transdermal administration comprise from about 125 μg to about 1250 mg, preferably from about 250 μg to about 500 mg, and more preferably from about 2.5 mg to about 250 mg, of the compounds admixed with a pharmaceutically acceptable carrier or diluent.

The invention also encompasses prodrugs of a compound of the invention which on administration undergo chemical conversion by metabolic or other physiological processes before becoming active pharmacological substances. Conversion by metabolic or other physiological processes includes without limitation enzymatic (e.g, specific enzymatically catalyzed) and non-enzymatic (e.g., general or specific acid or base induced) chemical transformation of the prodrug into the active pharmacological substance. In general, such prodrugs will be functional derivatives of a compound of the invention which are readily convertible in vivo into a compound of the invention. Conventional procedures for the selection and preparation of suitable prodrug derivatives are described, for example, in Design of prodrugs, ed. H. Bundgaard, Elsevier, 1985.

In another aspect, there are provided methods of making a composition of a compound described herein comprising formulating a compound of the invention with a pharmaceutically acceptable carrier or diluent. In some embodiments, the pharmaceutically acceptable carrier or diluent is suitable for oral administration. In some such embodiments, the methods may further comprise the step of formulating the composition into a tablet or capsule. In other embodiments, the pharmaceutically acceptable carrier or diluent is suitable for parenteral administration. In some such embodiments, the methods further comprise the step of lyophilizing the composition to form a lyophilized preparation.

B. Combinations. The compounds of the invention may be used in combination with i) one or more other NS3 protease inhibitors and/or ii) one or more other types of antiviral agents (employed to treat viral infection and related diseases) and/or one or more other types of therapeutic agents which may be administered orally in the same dosage form, in a separate oral dosage form (e.g., sequentially or non-sequentially) or by injection together or separately (e.g., sequentially or non-sequentially).

Accordingly, in another aspect the invention provides combinations, comprising:
a) a compound of the invention as described herein; and
b) one or more compounds comprising:
   i) other compounds of the present invention
   ii) anti-viral agents including, but not limited to, other NS3 protease inhibitors
   iii) anti-proliferative agents
   iv) immune modulators

Combinations of the invention can further comprise a pharmaceutically acceptable carrier. In some embodiments, the compound of the invention is 90 wt % or more of a single diastereomer or single enantiomer. Alternatively, the compound of the invention can be 91, 92, 93, 94, 95, 96, 97, 98, or 99 wt % or more of a single diastereomer or single enantiomer.

The dosages and formulations for the other antiviral agent to be employed, where applicable, will be as set out in the latest edition of the Physicians' Desk Reference.

In carrying out the methods of the invention, a composition may be employed containing the compounds of the invention, with or without another antiviral agent and/or other type therapeutic agent, in association with a pharmaceutical vehicle or diluent. The composition can be formulated employing conventional solid or liquid vehicles or diluents and pharmaceutical additives of a type appropriate to the mode of desired administration. The compounds can be administered to mammalian species including humans, monkeys, dogs, etc. by an oral route, for example, in the form of tablets, capsules, granules or powders, or they can be administered by a parenteral route in the form of injectable preparations. The dose for adult humans is preferably between 10 and 1,000 mg per day, which can be administered in a single dose or in the form of individual doses from 1-4 times per day.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

Definitions

The terms “HCV NS3 serine protease”, “HCV NS3 protease”, “NS3 serine protease”, and “NS3 protease” denote all active forms as well as mutant forms of the serine protease encoded by the NS3 region of the hepatitis C virus, including all combinations thereof with other proteins in either covalent or noncovalent association. For example, other proteins in this context include without limitation the protein encoded by the NS4a region of the hepatitis C virus. Accordingly, the terms “NS3/4a” and “NS3/4a protease” denote the NS3 protease in combination with the HCV NS4a protein.

The term “other type(s) of therapeutic agents” as employed herein refers to one or more antiviral agents (other than HCV NS3 serine protease inhibitors of the invention).

The term “treatment” is defined as the management and care of a patient for the purpose of combating the disease, condition, or disorder and includes administering a compound of the present invention to prevent the onset of the symptoms or complications, or alleviating the symptoms or complications, or eliminating the disease, condition, or disorder.

“Treating” within the context of the instant invention means an alleviation of symptoms associated with a disorder or disease, or inhibition of further progression or worsening of those symptoms, or prevention or prophylaxis of the disease or disorder. Similarly, as used herein, an “effective amount” or a “therapeutically effective amount” of a compound of the invention refers to an amount of the compound that alleviates, in whole or in part, symptoms associated with the disorder or condition, or halts further progression or worsening of those symptoms, or prevents or provides prophylaxis for the disorder or condition. A “therapeutically effective amount” refers to an amount effective, at dosages and for periods of time necessary, to achieve the desired therapeutic result by inhibition of HCV NS3 serine protease activity. A therapeutically effective amount is also one in which any toxic or detrimental effects of compounds of the invention are outweighed by the therapeutically beneficial effects. For example, in the context of treating HCV infection, a therapeutically effective amount of a HCV NS3 serine protease inhibitor of the invention is an amount sufficient to control HCV viral infection.

All chiral, diastereomeric, racemic forms of a structure are intended, unless the specific stereochemistry or isomeric form is specifically indicated. Compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

In general, “substituted” refers to an organic group as defined herein in which one or more bonds to a hydrogen atom contained therein are replaced by a bond to non-hydrogen or non-carbon atoms such as, but not limited to, a halogen (i.e., F, Cl, Br, and I); an oxygen atom in groups such as hydroxyl groups, alkoxy groups, aryloxy groups, aralkyloxy groups; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, N-oxides, hydrazides, azides, and enamines; and other heteroatoms in various other groups. Substituted alkyl, alkenyl, alkynyl, cycloalkyl, and cycloalkenyl groups as well as other substituted groups also include groups in which one or more bonds to a carbon(s) or hydrogen(s) atom are replaced by one or more bonds, including double or triple bonds, to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitrites.

Substituted ring groups such as substituted aryl, heterocyclyl and heteroaryl groups also include rings and fused ring systems in which a bond to a hydrogen atom is replaced with a bond to a carbon atom. Therefore, substituted aryl, heterocyclyl and heteroaryl groups may also be substituted with alkyl, alkenyl, and alkynyl groups as defined herein.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups may be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which may be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring maybe substituted with as many as N-1 substituents wherein N is the size of the carbocyclic ring with for example, amino, hydroxy, cyano, carboxy, nitro, thio, alkoxy, and halogen groups.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH3), —CH═C(CH3)2, —C(CH3)═CH2, —C(CH3)═CH(CH3), —C(CH2CH3)═CH2, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, cyclohexadienyl, butadienyl, pentadienyl, and hexadienyl groups.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH3), —C≡C(CH2CH3), —CH2C≡CH, —CH2C≡C(CH3), and —CH2C≡C(CH2CH3) among others.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halogen groups, bonded to one of the ring members. Rather, groups such as tolyl are referred to as substituted aryl groups. Representative substituted aryl groups may be mono-substituted or substituted more than once, such as, but not limited to, 2-, 3-, 4-, 5-, or 6-substituted phenyl or naphthyl groups, which may be substituted with groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. The phrase “heterocyclyl group” includes fused ring species including those comprising fused aromatic and non-aromatic groups. The phrase also includes polycyclic ring systems containing a heteroatom such as, but not limited to, quinuclidyl. However, the phrase does not include heterocyclyl groups that have other groups, such as alkyl or halogen groups, bonded to one of the ring members. Rather, these are referred to as “substituted heterocyclyl groups”. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups may be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups such as those listed above.

Additional examples of aryl and heteroaryl groups include but are not limited to phenyl, biphenyl, indenyl, naphthyl(1-naphthyl, 2-naphthyl), N-hydroxytetrazolyl, N-hydroxytriazolyl, N-hydroxyimidazolyl, anthracenyl(1-anthracenyl, 2-anthracenyl, 3-anthracenyl), thiophenyl(2-thienyl, 3-thienyl), furyl(2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl(2-pyrrolyl), pyrazolyl(3-pyrazolyl), imidazolyl(1-imidazolyl, 2-imidazolyl, 4-imidazolyl, 5-imidazolyl), triazolyl(1,2,3-triazol-1-yl, 1,2,3-triazol-2-yl 1,2,3-triazol-4-yl, 1,2,4-triazol-3-yl), oxazolyl(2-oxazolyl, 4-oxazolyl, 5-oxazolyl), thiazolyl(2-thiazolyl, 4-thiazolyl, 5-thiazolyl), pyridyl(2-pyridyl, 3-pyridyl, 4-pyridyl), pyrimidinyl(2-pyrimidinyl, 4-pyrimidinyl, 5-pyrimidinyl, 6-pyrimidinyl), pyrazinyl, pyridazinyl(3-pyridazinyl, 4-pyridazinyl, 5-pyridazinyl), quinolyl(2-quinolyl, 3-quinolyl, 4-quinolyl, 5-quinolyl, 6-quinolyl, 7-quinolyl, 8-quinolyl), isoquinolyl(1-isoquinolyl, 3-isoquinolyl, 4-isoquinolyl, 5-isoquinolyl, 6-isoquinolyl, 7-isoquinolyl, 8-isoquinolyl), benzo[b]furanyl(2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl(1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl(1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl(1-benzoxazolyl, 2-benzoxazolyl), benzothiazolyl(1-benzothiazolyl, 2-benzothiazolyl, 4-benzothiazolyl, 5-benzothiazolyl, 6-benzothiazolyl, 7-benzothiazolyl), carbazolyl(1-carbazolyl, 2-carbazolyl, 3-carbazolyl, 4-carbazolyl), 5H-dibenz[b,f]azepine(5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine(10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroaralkyl groups are alkyl groups as defined above in which a hydrogen or carbon bond of an alkyl group is replaced with a bond to a heteroaryl group as defined above.

The term “alkoxy” refers to an oxygen atom connected to an alkyl group as defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

The terms “aryloxy” and “arylalkoxy” refer to, respectively, an aryl group bonded to an oxygen atom and an aralkyl group bonded to the oxygen atom at the alkyl. Examples include but are not limited to phenoxy, naphthyloxy, and benzyloxy.

The term “alkanoyl”, alone or as part of another group, refers to alkyl linked to a carbonyl group.

The term “amine” (or “amino”) includes primary, secondary, and tertiary amines having, e.g., the formula —NR³⁰R³¹. R³⁰ and R³¹ at each occurrence are independently hydrogen, or a substituted or unsubstituted alkyl, alkenyl, alkynyl, cycloalkyl, cycloalkenyl, aryl, aralkyl, heterocyclyl or heterocyclylalkyl group as defined herein. Amines thus include but are not limited to —NH₂, alkylamines, dialkylamines, arylamines, alkylarylamines, diarylamines, aralkylamines, heterocyclylamines and the like.

The term “amide” (or “amido”) includes C— and N-amide groups, i.e., —C(O)NR³²R³³, and —NR³²C(O)R³³ groups, respectively. R³² and R³³ are independently hydrogen, or a substituted or unsubstituted alkyl, alkenyl, alkynyl, cycloalkyl, cycloalkenyl, aryl, aralkyl, heterocyclyl or heterocyclylalkyl group as defined herein. Amide groups therefore include but are not limited to carbamoyl groups (—C(O)NH₂) and formamide groups (—NHC(O)H).

The term “urethane” (or “carbamyl”) includes N— and O-urethane groups, i.e., —NR³⁴C(O)OR³⁵ and —OC(O)NR³⁴R³⁵ groups, respectively. R³⁴ and R³⁵ are independently hydrogen, or a substituted or unsubstituted alkyl, cycloalkyl, aryl, aralkyl, or heterocyclyl group as defined herein.

The term “sulfonamide” (or “sulfonamido”) includes S— and N-sulfonamide groups, i.e., —SO₂NR³⁶R³⁷ and —NR³⁶SO₂R³⁷ groups, respectively. R³⁶ and R³⁷ are independently hydrogen, or a substituted or unsubstituted alkyl, cycloalkyl, aryl, aralkyl, or heterocyclyl group as defined herein. Sulfonamide groups therefore include but are not limited to sulfamoyl groups (—SO₂NH₂).

The term “amidine” or “amidino” includes groups of the formula —C(NR³⁸)NR³⁹R⁴⁰. R³⁸, R³⁹, and R⁴⁰ are independently H, an amino protecting group, or a substituted or unsubstituted alkyl, cycloalkyl, aryl, aralkyl, or heterocyclyl group as defined herein. Typically, an amidino group is —C(NH)NH₂.

The term “guanidine” or “guanidino” includes groups of the formula —NR⁴¹C(NR⁴²)NR⁴³R⁴⁴. R⁴¹, R⁴², R⁴³, and R⁴⁴ are independently H, an amino protecting group, or a substituted or unsubstituted alkyl, cycloalkyl, aryl, aralkyl, or heterocyclyl group as defined herein. Typically, a guanidino group is —NHC(NH)NH₂.

The term “alkylene” denotes a divalent alkyl. Examples of alkylene include, without limitation, methylene, ethylene, propylene, and the like. The term “carboxyalkyl” includes groups of the formula —R⁴⁵—COOH wherein R⁴⁵ is a substituted or unsubstituted alkylene. The term “carboxamidoalkyl” includes groups of the formula —R⁴⁵—C(O)NR⁴³R⁴⁴ wherein R⁴⁵, R⁴³ and R⁴⁴ are as defined above. The term “heteroarylalkyl” includes groups of formula —R⁴⁵-heteroaryl, wherein R⁴⁵ and heteroaryl are as defined above. The term cycloalkylidenyl, alone or in combination with any other term, refers to a stable carbocyclic ring radical containing at least one exocyclic carbon-carbon double bond. Preferably, a cycloalkylidenyl has from 5-7 carbon atoms. Examples of cycloalkylidenyl include, without limitation, cyclopentylidenyl, cyclohexylidenyl, cyclopentenylidenyl, and the like. The term heterocycloalkylidenyl, alone or in combination with any other term, refers to a stable heterocyclic ring radical containing at least one exocyclic carbon-carbon double bond.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### Examples

A further detailed description of the invention is given with reference to the following non-limiting examples.

### Example 1

### Synthesis of 2-{[1-(2-Benzyloxycarbonylamino-3-methyl-butyryl)-4-(2-phenyl-quinolin-4-yloxy)-pyrrolidine-2-carbonyl]-amino}-butyric boronate (+)-pinanediol ester. (6)

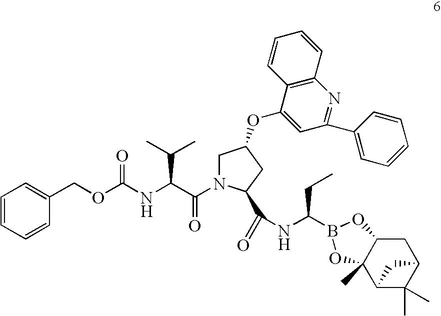

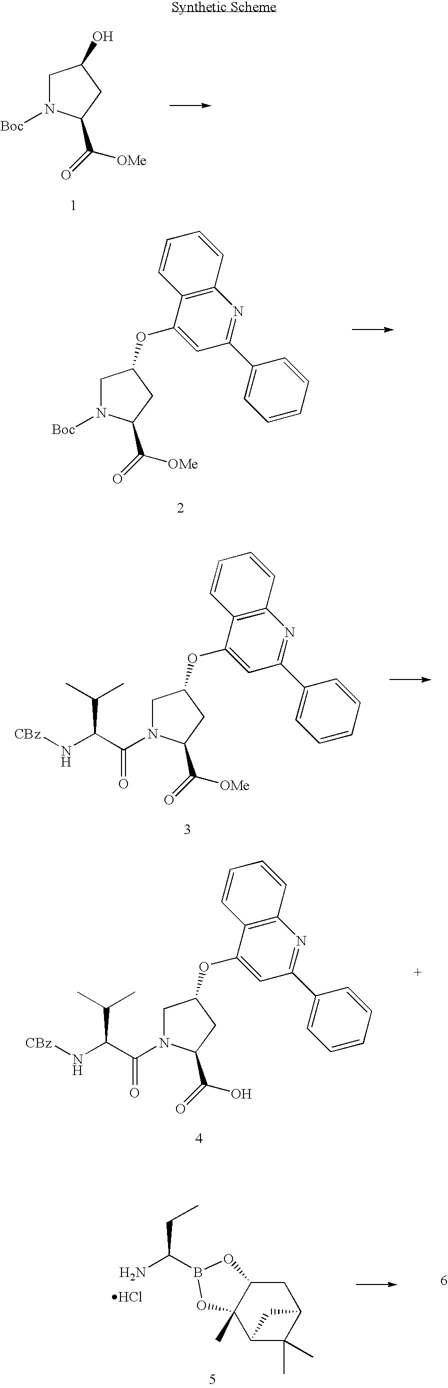

### Synthesis of 2; (4-(2-Phenylquinolin-4-yloxy)-pyrrolidine-1,2-dicarboxylic acid 1-tert-butyl ester-2-methyl ester)

To a stirred solution of compound 1 (boc-cis-4-hydroxyproline-OMe, 490 mg, 2.0 mmol), 2-phenyl-quinolin-4-ol (synthesized as described in J. Med. Chem. 2004, 47, 123) (442 mg, 2.0 mmol), and triphenylphosphine (1.050 g, 4.0 mmol) in anhydrous THF (30 mL) cooled to 0° C. was added diisopropylazodicarboxylate (0.808 g, 4.0 mmol, 774 μL) drop-wise. The resulting yellow solution was warmed to RT and allowed to react for an additional 48 h. The reaction solution was concentrated in vacuo and purified by flash column chromatography (silica gel, gradient of EtOAc/hex [30-60% EtOAc]) to give 4-(2-Phenyl-quinolin-4-yloxy)-pyrrolidine-1,2-dicarboxylic acid 1-tert-butyl ester 2-methyl ester (2) as a yellow solid (544 mg, yield 60%). MS m/z (rel intensity) 449 [M+1] (5), 393 (31), 222 (100).

### Synthesis of 3; (1-(2-Benzyloxycarbonylamino-3-methyl-butyl)-4-(2-phenyl-quinolin-4-yloxy)-pyrrolidine-2-carboxylic acid methyl ester)

To a solution of compound 2 dissolved in minimal DCM was added 4N HCl in dioxane (3 mL). The reaction was stirred at rt for 30 min followed by concentration in vacuo to provide 4-(2-Phenyl-quinolin-4-yloxy)-pyrrolidine-2-carboxylic acid methyl ester HCl salt. This white solid was thoroughly dried and used in the next step without firther purification.

To an ice cooled solution of 4-(2-Phenyl-quinolin-4-yloxy)-pyrrolidine-2-carboxylic acid methyl ester HCl (200 mg, 0.52 mmol), Z-Val-OH (155 mg, 0.62 mmol), EDAC (148 mg, 0.78 mmol), and HOBt (105 mg, 0.78 mmol) in 5 mL of anhydrous CH₂Cl₂ was added NMM (157 mg, 1.56 mmol) drop wise. The reaction is allowed to warm to room temperature under a blanket of N₂ for 18 h. The reaction was quenched with addition of with NaHCO₃ saturated solution. The organic phase was removed and the water layer was extracted with additional DCM (2×25 mL). The organic layers were combined, washed with brine, dried over MgSO₄, and concentrated in vacuo. The crude product was purified via flash column chromatography (silica gel, gradient of 30%-60% EtOAc/hex) to provide compound 3 as a white solid (180 mg, 0.31 mmol, 60%).

### Synthesis of 4; (1-(2-Benzyloxycarbonylamino-3-methyl-butyryl)-4-(2-phenyl-quinolin-4yloxy)-pyrrolidine-2-carboxylic acid)

To a solution of compound 3 (90 mg, 0.15 mmol) in 1:1 THF/H₂O (10 mL) was added LiOH.H₂O (13 mg, 0.30 mmol). The solution is stirred for 2 h at room temperature. The THF was removed and the aqueous layer was acidified by addition of 1 N HCl (300 μL, 0.30 mmol) and the resulting white cloudy solution was extracted with EtOAc (3×5 mL). The organics layers were combined, washed with brine, dried over MgSO₄, and concentrated in vacuo to give the desired acid 4 as a white solid (77 mg, 0.135 mmol, 90%).

### Synthesis of 6; 2-{[1-(2-Benzyloxycarbonylamino-3-methyl-butyryl)-4-(2-phenyl-quinolin-4-yloxy)-pyrrolidine-2-carbonyl]-amino}-butyric boronate (+)-pinanediol ester

To an ice cooled solution of compound 4 (425 mg, 0.75 mmol) in anhydrous DCM (10 mL) was added HOBt (151 mg, 1.125 mmol, 1.5 eq.), EDAC (215 mg, 1.125 mmol, 1.5 eq). The solution was stirred for 10 min followed by addition of 5 (478 mg, 1.875 mmol, 2.0 eq) (synthesized as described in Organometallics 1984, 3, 1284; Tetrahedron 2003, 59, 579) and NMM (0.38 mL, 2.25 mmol). The resulting yellow solution was allowed to warm to room temperature overnight. The reaction was quenched by addition of NaHCO₃ saturated solution. The organic phase was removed and the water layer was extracted with additional DCM (2×25 mL). The organic layers were combined, washed with brine, dried over MgSO₄, and concentrated in vacuo. The crude resultant was further purified via flash column chromatography (silica gel, 2% MeOH in DCM) to afford 6 as a white solid (20 mg, 0.025 mmol). MS m/z (rel intensity) 788 [M+1] (10), 566 (100).

The following compounds were synthesized according to the synthesis described for example 1:

| No. | Structure | LC-MS |
|---|---|---|
| 01 | 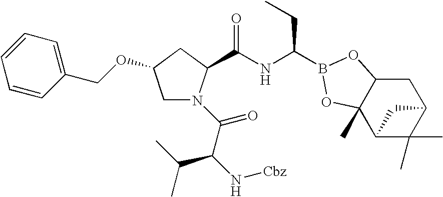 | 697 [M + 23] (9), 675 [M + 1] (100) |
| 6 | 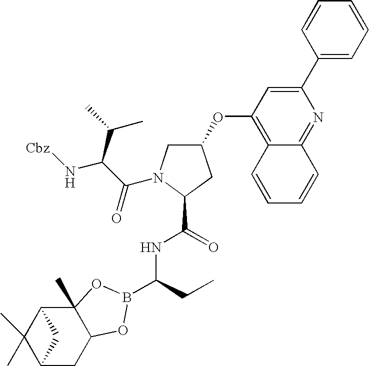 | 788 [M + 1] (10), 566 (100) |
| 33 | 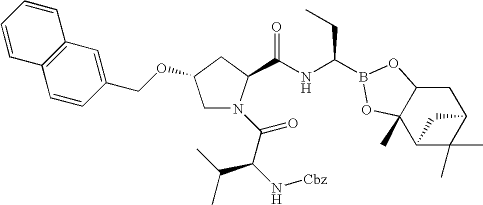 | 746 [M + 23] (16), 724 [M + 1] (55), 572 (100) |
| 35 | 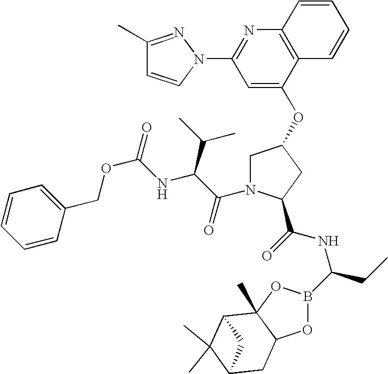 | 813 [M + 23] (9), 791 [M + 1] (41), 566 (100) |
| 36 | 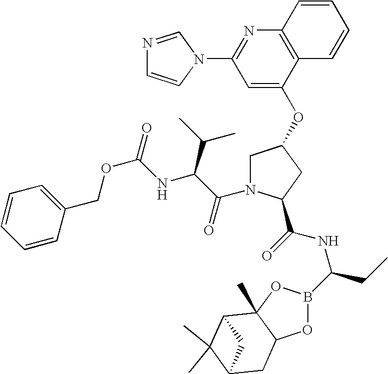 | 799 [M + 23] (4), 777 [M + 1] (100), 625 (49) |
| 57 | 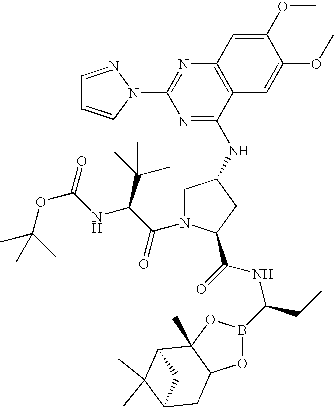 | 818 [M + 1] (100), 718 (36) |

### Example 2

### Synthesis of 4-(2-Phenyl-quinolin-4-yloxy)-2-[1-(2,9,9-trimethyl-3,5-dioxa-4-bora-tricyclo[6.1.1.0^{2,6}]dec-4-yl)-propylcarbamoyl]-pyrrolidine-1-carboxylic acid benzyl ester (10)

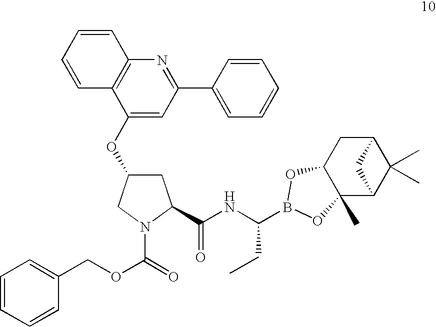

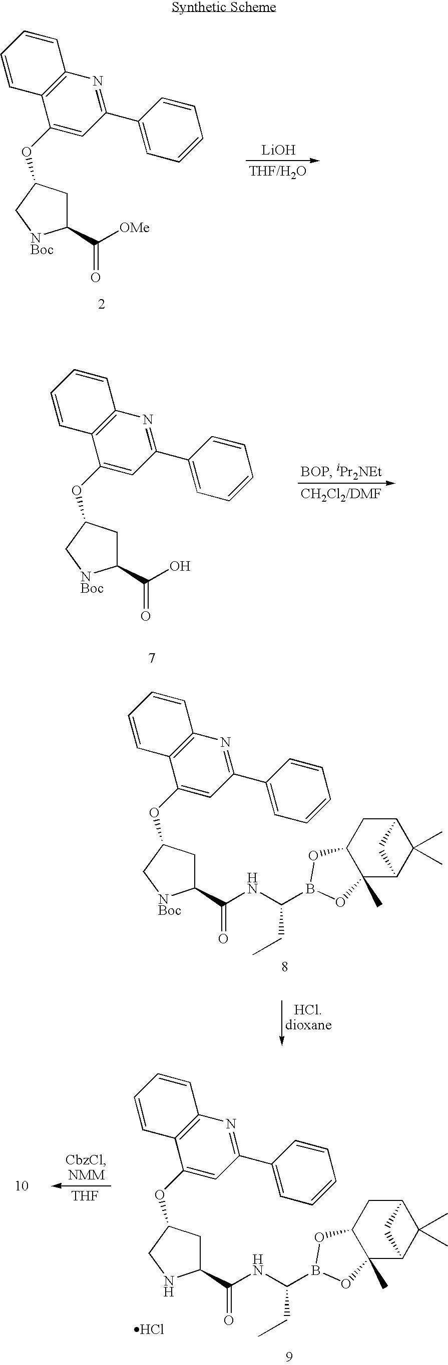

### Synthesis of 7; 4-(2-Phenyl-quinolin-4-yloxy)-pyrrolidine-1,2-dicarboxylic acid 1-tert-butyl ester

Compound 2 (1.78 g, 3.97 mmol) was treated with LiOH monohydrate (167 mg, 3.97 mmol) in a way similar to that described for the synthesis of 4 to yield 7 (950 mg, 2.19 mmol). MS m/z (rel intensity) 435 [M+1] (4), 379 (47), 222 (100).

### Synthesis of 8; 4-(2-Phenyl-quinolin-4-yloxy)-2-[1-(2,9,9-trimethyl-3,5-dioxa-4-bora-tricyclo[6.1.1.0^{2,6}]dec-4-yl)-propylcarbamoyl]-pyrrolidine-1-carboxylic acid tert-butyl ester

To a solution of 7 (250 mg, 0.58 mmol) in a 4:1 mixture of CH₂Cl₂:DMF (2.3 mL) was added BOP (280 mg, 0.63 mmol). After cooling to −10° C., ⁱPr₂EtN (0.1 mL, 0.58 mmol) was added dropwise. After being stirred for 10 min, the mixture was allowed to warm to room temperature for 1 h. After cooling to −15° C., 1-(2,9,9-Trimethyl-3,5-dioxa-4-bora-tricyclo[6.1.1.0^{2,6}]dec-4-yl)-ethylamine hydrochloride (283 mg, 1.04 mmol) was added in one portion followed by dropwise addition of a solution of ⁱPr₂EtN (281 uL, 1.6 mmol) in CH₂Cl₂ (0.46 ml). The mixture was stirred at −10° C. for 1 h and then allowed to warm to room temperature. After 15 min, the mixture was diluted with EtOAc and washed with citric acid (5% aqueous solution), brine, saturated aqueous NaHCO₃ solution, and brine. The organic phase was dried over Na₂SO₄, filtered, and evaporated under reduced pressure. The residue was purified by silica gel column chromatography to afford 8 (270 mg, 0.41 mmol) as a white solid. MS m/z (rel intensity) 654 [M+1] (4), 433 (99), 377 (100).

### Synthesis of 9; 4-(2-Phenyl-quinolin-4-yloxy)-pyrrolidine-2-carboxylic acid [1-(2,9,9-trimethyl-3,5-dioxa-4-bora-tricyclo[6.1.1.0^{2,6}]dec-4-yl)-propyl]-amide hydrochloride

Compound 3 (750 mg, 1.1 mmol) was treated with a 4N solution of HCl in 1,4-dioxane (20 mL). The reaction was stirred at 0° C. for 10 min and then allowed to warm up to room temperature. After being stirred for 2 h, the solvent was removed under reduced pressure to afford compound 9 (650 mg, 1.1 mmol).

### Synthesis of 10; 4-(2-Phenyl-quinolin-4-yloxy)-2-[1-(2,9,9-trimethyl-3,5-dioxa-4-bora-tricyclo[6.1.1.0^{2,6}]dec-4-yl)-propycarbamoyl]-pyrrolidine-1-carboxylic acid benzyl ester

To a suspension of 9 (62 mg, 0.11 mmol) in THF (2 mL) was added N-methyl morpholine (23 μL, 0.21 mmol). The resulting mixture was cooled to 0° C. and benzyl chloroformate (18 μL, 0.13 mmol) was added dropwise. After being stirred at 0° C. for 10 min, the mixture was allowed to warm to room temperature. After being stirred for three hours, the mixture was diluted with EtOAc (3 ml) and washed with citric acid (5% aqueous solution), brine, sodium saturated aqueous NaHCO₃ solution, and brine. The organic phase was dried over Na₂SO₄, filtered and concentrated under reduced pressure. The oily residue was purified by silica gel column chromatography to afford compound 10 (46 mg, 0.07 mmol) as a white solid. MS m/z (rel intensity) 710 [M+23] (5), 688 [M+1] (10), 467 (100).

The following compounds were synthesized according to the synthesis described for example 2:

| No. | Structure | LC-MS |
|---|---|---|
| 53 | 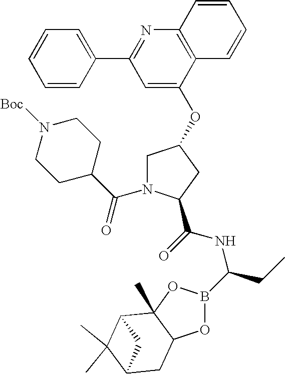 | 766 [M + 1] (34), 488 (100) |
| 56 | 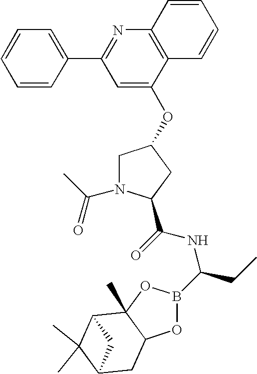 | 596 [M + 1] (8), 375 (100) |
| 58 | 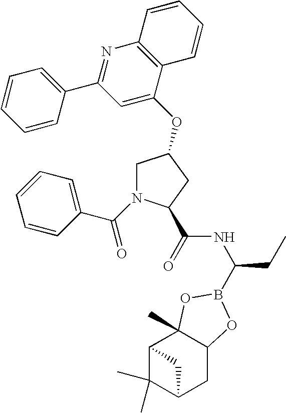 | 658 [M + 1] (6), 437 (100) |
| 59 | 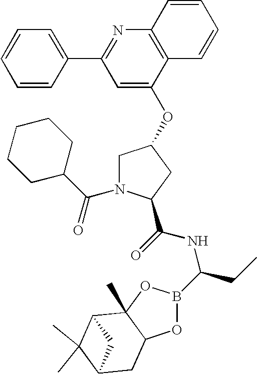 | 664 [M + 1] (8), 443 (100) |
| 10 | 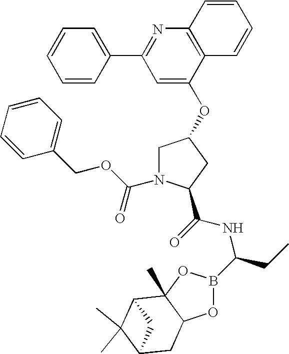 | 710 [M + 23] (5), 688 [M + 1] (10), 467 (100) |
| 22 | 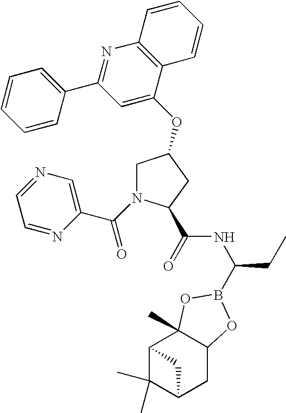 | 660 [M + 1] (7), 439 (100) |
| 24 | 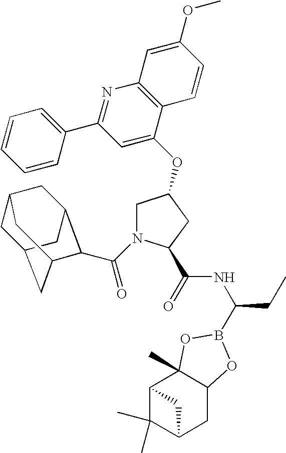 | 747 [M + 1] (18), 495 (100) |

### Example 3

### HCV-NS3/4a Protease Assay

### Materials

HCV NS3/4a of genotype 1b, 5-FAM/QXL520 fluorescence resonance energy transfer (FRET) peptide, and buffer were purchased from Anaspec, San Jose. The sequence of this FRET peptide is derived from the cleavage site of NS4a/NS4b. IC_{50/90} calculations were performed by non-linear regression analysis using Prism software (GraphPad).

### Methods

Biochemical assay. Either 5 μL of DMSO or 5 μL of compound solution in DMSO at various concentrations were added to 45 μL of buffer containing 5 ng of NS3/4a per well in a 96 well plates for “enzyme only” and “compound testing” wells. “No enzyme” wells contain 45 μL of reaction buffer without the enzyme and 5 μL of DMSO. Plates were preincubed at room temperature for 1 hour. Protease reaction was initiated by addition of 50 μL of NS3/4a protease substrate solution to give a final concentration of 2 μM. After shaking gently for 60 second and incubating at room temperature for 5 min, each well was measured for fluorescence intensity at Ex/Em=490 nm/520 nM every 5 minutes for 30 min. IC₅₀ and IC₉₀ values were calculated by non-linear regression analysis using Prism software (GraphPad).

While the invention has been described and exemplified in sufficient detail for those skilled in this art to make and use it, various alternatives, modifications, and improvements will be apparent to those skilled in the art without departing from the spirit and scope of the claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

Other embodiments are set forth within the following claims.

1. A compound of Formula I: 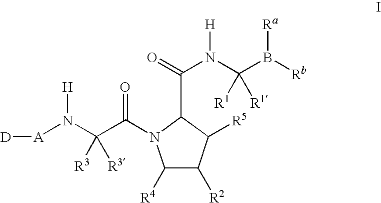
and stereoisomers, solvates, hydrates, tautomers, prodrugs, pharmaceutically acceptable salts, and mixtures thereof,
wherein:
A is a bond, —C(O)—, —C(O)O—, —C(O)NH—, —S(O)—, or —S(O)₂—;
D is —R⁶, -alkyl-R⁶, -alkenyl-R⁶, -alkynyl-R⁶, —OR⁶, —NHR⁶, —N(R⁶)₂, or —C(O)R⁶;
R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; provided that R¹ or R^{1′} is not 2,2-difluoroethyl; or
R¹ and R¹′ together with the carbon atom to which they are attached form a 3-7 membered substituted or unsubstituted carbocycle;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
when R¹, R¹′ and R⁴ are not hydrogen, then
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
when R¹ and R¹′ are not hydrogen, and R⁴is hydrogen, then
R² is —(CH₂)ₓ—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷′, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH2)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
when either R¹ or R¹′ is hydrogen, and R⁴ is not hydrogen, then
R² is —(CH₂)ₓ—R⁸′, —(CH₂)ₓ—NH(R⁹)—R⁸, —(CH₂)_{w}—O—R⁸′, —(CH₂)ₓ—S—R⁸, —O—(CH₂)_{w}—R⁸′, —NH(R⁹)—(CH₂)ₓ—R⁸′, —S—(CH₂)ₓ—R⁸, —O—R⁸′, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸;
when either R¹ or R¹′ is hydrogen, and R⁴ is hydrogen, then
R² is —(CH₂)ₓ—R⁸′, —(CH₂)ₓ—NH(R⁹)—R⁸, —(CH₂)_{w}—O—R⁸′, —(CH₂)ₓ—S—R⁸, —O—(CH₂)_{w}—R⁸′, —NH(R⁹)—(CH₂)ₓ—R⁸′, —S—(CH₂)ₓ—R⁸, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸;
R⁶ is a substituted or unsubstituted aryl, heteroaryl, cycloalkyl, heterocyclyl, cycloalkylidenyl, or heterocycloalkylidenyl group;
R⁷ and R¹¹ are independently a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁷′ is a substituted or unsubstituted alkyl, cycloalkyl, cycloalkenyl, cycloalkylalkyl, cycloalkenylalkyl, saturated or unsaturated heterocyclyl, saturated or unsaturated heterocyclylalkyl group, or an aryl or heteroaryl group substituted with NR¹⁰R¹¹;
R⁸ is a divalent substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R⁸′ is a divalent substituted or unsubstituted cycloalkyl, cycloalkenyl, cycloalkylalkyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R⁹ and R¹⁰ are independently hydrogen or R⁷;
w is in the range 1-6; and
x is in the range 0-6.

2. The compound of claim 1 wherein:
R¹ and R¹′ are independently a substituted or unsubstituted alkyl, alkenyl, alkynyl, cycloalkyl, cycloalkylalkyl, or arylalkyl group; provided that R¹ or R^{1′} is not 2,2-difluoroethyl; or
R¹ and R¹′ together with the carbon atom to which they are attached form a 3-7 membered substituted or unsubstituted carbocycle;
R⁴ is a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group; and
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷.

3. The compound of claim 2 wherein R² is —O—(CH₂)_{w}—R⁷ or —O—R⁷.

4. The compound of claim 1 wherein:
R¹ and R¹′ are independently a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R⁴ is hydrogen; and
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷′, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH2)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷.

5. The compound of claim 4 wherein R² is —O—(CH₂)_{w}—R⁷ or —O—R⁷′.

6. The compound of claim 5 wherein R⁷is a substituted or unsubstituted aryl or heteroaryl, wherein the number of substituents, if present, is in the range 1-3.

7. The compound of claim 1 wherein:
either R¹ or R¹′ is hydrogen;
R⁴ is not hydrogen; and
R² is —(CH₂)ₓ—R⁸′, —(CH₂)ₓ—NH(R⁹)—R⁸, —(CH₂)_{w}—O—R⁸′, —(CH₂)ₓ—S—R⁸, —O—(CH₂)_{w}—R⁸′, —NH(R⁹)—(CH₂)ₓ—R⁸′, —S—(CH₂)ₓ—R⁸′, —O—R⁸′, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸.

8. The compound of claim 7 wherein R2 is —O—(CH₂)_{w}—R⁸′ or —O—R⁸′.

9. The compound of claim 8 wherein R⁸′ is a divalent substituted or unsubstituted aryl or heteroaryl group, each of which is attached to a substituted or unsubstituted heteroaryl group.

10. The compound of claim 1 wherein:
either R¹ or R¹′ is hydrogen;
R⁴ is hydrogen; and
R² is —(CH₂)ₓ—R⁸′, —(CH₂)ₓ—NH(R⁹)—R⁸, —(CH₂)_{w}—O—R⁸′, —(CH₂)ₓ—S—R⁸, —O—(CH₂)_{w}—R⁸′, —NH(R⁹)—(CH₂)ₓ—R⁸′, —S—(CH₂)ₓ—R⁸, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸.

11. The compound of claim 10 wherein R² is —O—(CH₂)_{w}—R⁸′.

12. The compound of claim 11 wherein R⁸′ is a divalent substituted or unsubstituted aryl or heteroaryl group, each of which is attached to a substituted or unsubstituted heteroaryl group.

13. The compound of claim 1 wherein R³ and R³′ are independently hydrogen or a substituted or unsubstituted alkyl group, provided, however, that at least one of R³ and R³′ is hydrogen.

14. The compound of claim 1 wherein A is selected from the group consisting of —C(O)— and —C(O)O—.

15. The compound of claim 1 wherein D is R⁶ or -alkyl-R⁶.

16. The compound of claim 15 wherein R⁶ is a substituted or unsubstituted aryl, or heteroaryl group.

17. The compound of claim 1 wherein R⁹ is hydrogen.

18. A compound of Formula II: 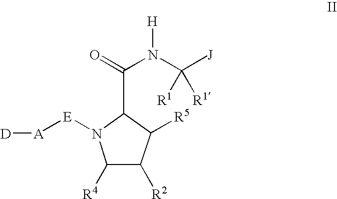
and stereoisomers, solvates, hydrates, tautomers, prodrugs, pharmaceutically acceptable salts, and mixtures thereof,
wherein:
J is 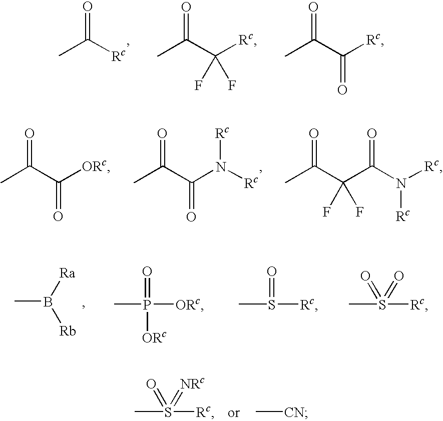
R^{c} at each occurrence is independently hydrogen, substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroararalyl; or
two R^{c} groups which are bound to the same nitrogen atom form together with said nitrogen atom a 5-7 membered monocylic heterocyclic ring system;
A is a bond, —C(O)—, —C(O)O—, —C(O)NH—, —S(O)—, or —S(O)₂;
D is —R⁶, -alkyl-R⁶, -alkenyl-R⁶, -alkynyl-R⁶, —OR⁶, —NHR⁶, —N(R⁶)₂, or —C(O)R⁶;
E is a bond or 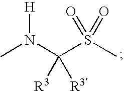
R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; provided that R¹ or R¹′ is not 2,2-difluoroethyl; or
R¹ and R¹′ together with the carbon atom to which they are attached form a 3-7 membered substituted or unsubstituted carbocycle;
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
R⁶ is a substituted or unsubstituted aryl, heteroaryl, cycloalkyl, heterocyclyl, cycloalkylidenyl, or heterocycloalkylidenyl group;
R⁷ at each occurrence is independently a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁹ is hydrogen or R⁷;
w is in the range 1-6; and
x is in the range 0-6.

19. The compound of claim 18 with structure of Formula IIa:

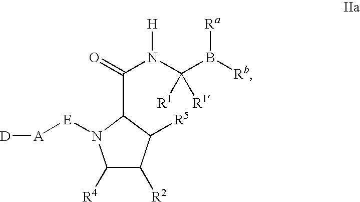

20. The compound of claim 18 wherein R² is —O—(CH₂)_{w}—R⁷.

21. The compound of claim 20 wherein R⁷ is a substituted or unsubstituted aryl or heteroaryl, wherein the number of substituents, if present, is in the range 1-3.

22. The compound of claim 18 wherein A is —C(O)— or —C(O)O—.

23. The compound of claim 18 wherein D is R⁶ or -alkyl-R⁶.

24. The compound of claim 23 wherein R⁶ is a substituted or unsubstituted aryl, or heteroaryl group.

25. The compound of claim 18 with structure of Formula IIb:

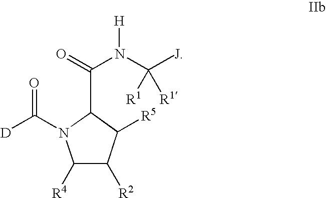

26. The compound of claim 18 with structure of Formula IIc:

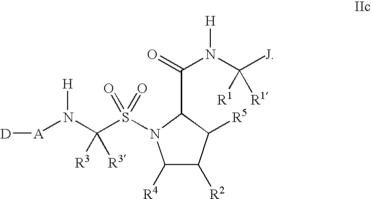

27. The compound of claim 26 wherein R³′ is hydrogen.

28. The compound of claim 18 with structure of Formula IId:

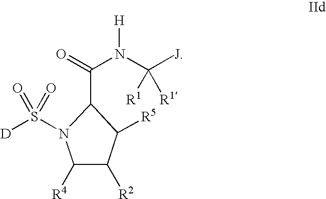

29. A compound of Formula III: 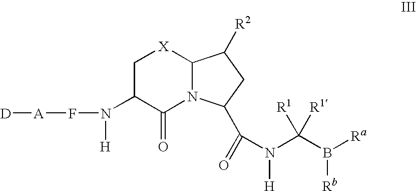
and stereoisomers, solvates, hydrates, tautomers, prodrugs, pharmaceutically acceptable salts, and mixtures thereof,
wherein:
X is CH₂ or S;
A is a bond, —C(O)—, —C(O)O—, —C(O)NH—, —S(O)—, or —S(O)₂—;
D is —R⁶, -alkyl-R⁶, -alkenyl-R⁶, -alkynyl-R⁶, —OR⁶, —NHR⁶, —N(R⁶)₂, or —C(O)R⁶;
F is a bond or 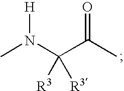
R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; or
R¹ and R¹′ together with the carbon atom to which they are attached form a 3-7 membered substituted or unsubstituted carbocycle;
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁶ is a substituted or unsubstituted aryl, heteroaryl, cycloalkyl, heterocyclyl, cycloalkylidenyl, or heterocycloalkylidenyl group;
R⁷ is a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁹ is hydrogen or R⁷;
w is in the range 1-6; and
x is in the range 0-6.

30. The compound of claim 29 wherein R² is —O—(CH₂)_{w}—R⁷.

31. The compound of claim 30 wherein R⁷ is a substituted or unsubstituted aryl or heteroaryl, wherein the number of substituents, if present, is in the range 1-3.

32. The compound of claim 29 wherein A is selected from the group consisting of —C(O)— and —OC(O)—.

33. The compound of claim 29 wherein D is R⁶ or -alkyl-R⁶.

34. The compound of claim 33 wherein R⁶ is a substituted or unsubstituted aryl, or heteroaryl group.

35. The compound of claim 29 wherein R⁹ is hydrogen.

36. The compound of claim 29 with structure of Formula IIIa:

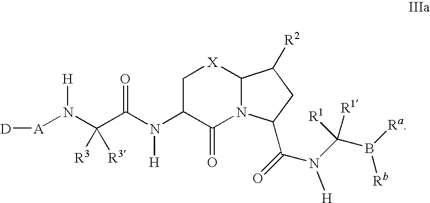

37. The compound of claim 36 wherein R³′ is hydrogen.

38. The compound of claim 29 with structure of Formula IIIb:

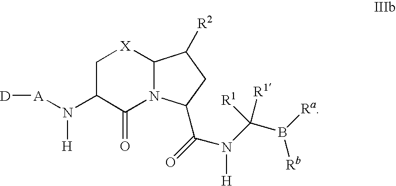

39. A compound of Formula IV: 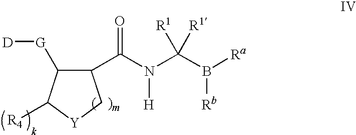
and stereoisomers, solvates, hydrates, tautomers, prodrugs, pharmaceutically acceptable salts, and mixtures thereof,
wherein:
D is —R⁶, -alkyl-R⁶, -alkenyl-R⁶, -alkynyl-R⁶, —OR⁶, —NHR⁶, —N(R⁶)₂, or —C(O)R⁶;
G is 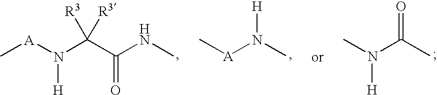
A is a bond, —C(O)—, —C(O)O—, —C(O)NH—,—S(O)—, —S(O)₂—;
Y is selected from the group consisting of O, S, CHR², and NR²′; such that
when Y is CHR², then k is in the range 0-3, and m is in the range 0-3;
when Y is NR²′, then k is in the range 1-3, and m is in the range 0-3; and
when Y is O or Y is S, then k is in the range 1-3, and m is in the range 0-3;
R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R¹ and R¹′ are independently hydrogen or a substituted or unsubstituted alkyl, alkenyl, alkynyl, cycloalkyl, cycloalkylalkyl, or arylalkyl group; provided that R¹ or R^{1′} is not 2,2-difluoroethyl; or
R¹ and R¹′ together with the carbon atom to which they are attached form a 3-7 membered substituted or unsubstituted carbocycle;
R² is —(CH₂)ₓ—R⁷, —(CH₂)ₓ—NH(R⁹)—R⁷, —(CH₂)_{w}—O—R⁷, —(CH₂)ₓ—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R²′ is —(CH₂)ₓ—R⁷, —(CH₂)_{z}—NH(R⁹)—R⁷, —(CH₂)_{z}—O—R⁷, —(CH₂)_{z}—S—R⁷, —O—(CH₂)_{w}—R⁷, —NH(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ is hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
R⁶ is a substituted or unsubstituted aryl, heteroaryl, cycloalkyl, heterocyclyl, cycloalkylidenyl, or heterocycloalkylidenyl group;
R⁷ is a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁹ is hydrogen or R⁷;
w is in the range 1-6;
x is in the range 0-6; and
z is in the range 2-6.

40. The compound of claim 39 with structure of Formula IVa:

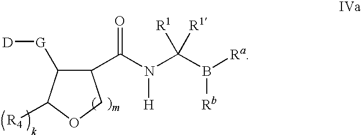

41. The compound of claim 39 with structure of Formula IVb:

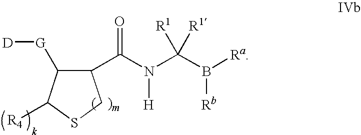

42. The compound of claim 39 with structure of Formula IVc:

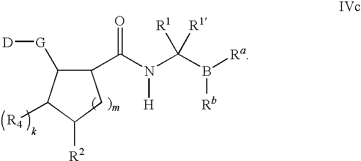

43. The compound of claim 42 wherein R² is —O—(CH₂)_{w}—R⁷.

44. The compound of claim 43 wherein R⁷ is a substituted or unsubstituted aryl or heteroaryl, wherein the number of substituents, if present, is in the range 1-3.

45. The compound of claim 39 with structure of Formula IVd:

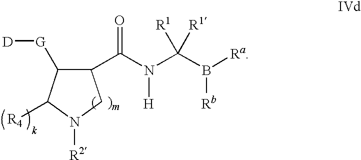

46. The compound of claim 45 wherein R² is —O—(CH₂)_{w}—R⁷.

47. The compound of claim 46 wherein R⁷ is a substituted or unsubstituted aryl or heteroaryl, wherein the number of substituents, if present, is in the range 1-3.

48. The compound of claim 39 with structure of Formula IVe:

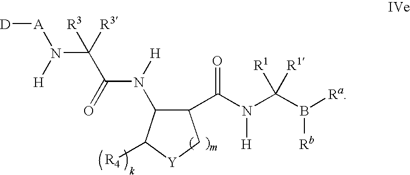

49. The compound of claim 48 wherein R³′ is hydrogen.

50. The compound of claim 48 wherein A is a bond, —C(O)—, or —C(O)O—.

51. The compound of claim 39 with structure of Formula IVf:

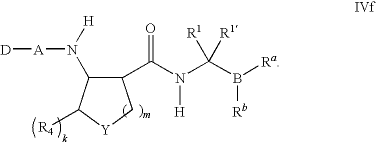

52. The compound of claim 51 wherein A is a bond, —C(O)—, or —C(O)O—.

53. The compound of claim 39 with structure of Formula IVg:

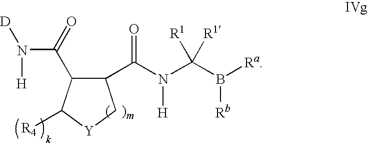

54. The compound of claim 39 wherein D is R⁶ or -alkyl-R⁶.

55. The compound of claim 54 wherein R⁶ is a substituted or unsubstituted aryl, or heteroaryl group.

56. A compound of Formula V: 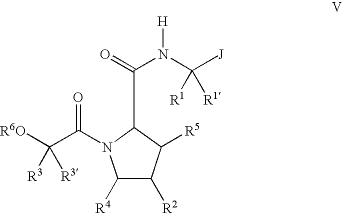
and stereoisomers, solvates, hydrates, tautomers, prodrugs, pharmaceutically acceptable salts, and mixtures thereof,
wherein:
J is 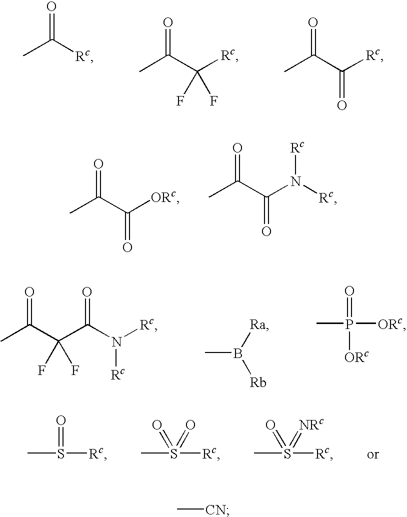
R^{a} and R^{b} are independently a hydroxyl or a group that can be hydrolyzed to hydroxyl, or R^{a} and R^{b} together with the boron to which they are attached form a cyclic group which can be hydrolyzed to B(OH)₂;
R^{c} at each occurrence is independently hydrogen, substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroararalyl; or
two R^{c} groups which are bound to the same nitrogen atom form together with said nitrogen atom a 5-7 membered monocylic heterocyclic ring system;
R¹ and R¹′ are independently hydrogen or a substituted or unsubstituted alkyl, alkenyl, alkynyl, cycloalkyl, cycloalkylalkyl, or arylalkyl group; provided that R¹ or R¹′ is not 2,2-difluoroethyl; or
R¹ and R¹′ together with the carbon atom to which they are attached form a 3-7 membered substituted or unsubstituted carbocycle;
R³ and R³′ are independently hydrogen or a substituted or unsubstituted alkyl, cycloalkyl, cycloalkylalkyl, aryl, aralkyl, heterocyclyl, heterocyclylalkyl, heteroaryl, heteroaralkyl, carboxyalkyl, or carboxamidoalkyl group, provided, however, that at least one of R³ and R³′ is not hydrogen;
R⁴ and R⁵ are independently hydrogen or a substituted or unsubstituted alkyl, alkenyl, aryl, aralkyl, aralkenyl, cycloalkyl, cycloalkylalkyl, cycloalkenyl, heterocyclyl, heterocyclylalkyl, heteroaryl, or heteroarylalkyl group;
provided that when R¹ and R¹′ are not hydrogen, then
R² is —(CH₂)ₓ—R⁷, —(CH₂)ₓ—N(R⁹)—R⁷, —(CH₂)_{w}—O—R⁷, —(CH₂)ₓ—S—R⁷, —O—(CH₂)_{w}—R⁷, —N(R⁹)—(CH₂)ₓ—R⁷, —S—(CH₂)ₓ—R⁷, —O—R⁷, —NH—R⁷, —S—R⁷, —NR⁹R⁷, —NHC(O)—(CH₂)ₓ—R⁷, —C(O)NH—(CH₂)ₓ—R⁷, —NHC(O)NH—(CH₂)ₓ—R⁷, —OC(O)NH—(CH₂)ₓ—R⁷, —NHC(O)O—(CH₂)ₓ—R⁷, —(CH₂)ₓ—NHC(O)—R⁷, —(CH₂)ₓ—C(O)NH—R⁷, —(CH₂)ₓ—NHC(O)NH—R⁷, —(CH₂)ₓ—OC(O)NH—R⁷, or —(CH₂)ₓ—NHC(O)O—R⁷; or R² and R⁴ together with the carbon atoms to which they are attached form a fused 3-6 member substituted or unsubstituted carbocyclyl or heterocyclyl group;
provided that when either R¹ or R¹′ is hydrogen, then
R² is —(CH₂)ₓ—R⁸′, —(CH₂)ₓ—N(R⁹)—R⁸, —(CH₂)_{w}—O—R⁸′, —(CH₂)ₓ—S—R⁸, —O—(CH₂)_{w}—R⁸′, —N(R⁹)—(CH₂)ₓ—R⁸′, —S—(CH₂)ₓ—R⁸, —O—R⁸′, —NH—R⁸′, —S—R⁸′, —NR⁹R⁸′, —NHC(O)—(CH₂)ₓ—R⁸, —C(O)NH—(CH₂)ₓ—R⁸′, —NHC(O)NH—(CH₂)ₓ—R⁸′, —OC(O)NH—(CH₂)ₓ—R⁸′, —NHC(O)O—(CH₂)ₓ—R⁸′, —(CH₂)_{w}—NHC(O)—R⁸, —(CH₂)ₓ—C(O)NH—R⁸′, —(CH₂)ₓ—NHC(O)NH—R⁸, —(CH₂)_{w}—OC(O)NH—R⁸, or —(CH₂)_{w}—NHC(O)O—R⁸; or R² and R⁵ together with the carbon atoms to which they are attached form a fused 3-6 member substituted or unsubstituted carbocyclyl or heterocyclyl group;
R⁶ is hydrogen or a substituted or unsubstituted alkyl, alkenyl, alkynyl, aryl, heteroaryl, cycloalkyl, heterocyclyl, cycloalkylidenyl, or heterocycloalkylidenyl group;
R⁷ at each occurrence is independently a substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group;
R⁸ is a divalent substituted or unsubstituted alkyl, cycloalkyl, cycloalkenyl, cycloalkylalkyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R⁸′ is a divalent substituted or unsubstituted cycloalkyl, cycloalkenyl, cycloalkylalkyl, cycloalkenylalkyl, aryl, aralkyl, heterocyclyl, heterocyclylalkyl, heteroaryl, or heteroarylalkyl group, each of which is attached to a substituted or unsubstituted heteroaryl group, wherein the number of substitutions, if present, is 1-3;
R⁹ is hydrogen or R⁷;
w is in the range 1-6; and
x is in the range 0-6.

57. The compound of claim 56
wherein
J is

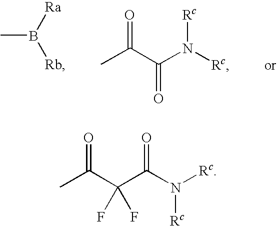

58. The compound of claim 56 with structure of Formula Va:

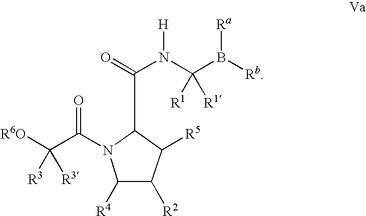

59. The compound of any one of claims 1, 18, 29, 39 or 56 wherein R^{a} and R^{b} are independently hydroxyl, methoxy, ethoxy, n-propoxy, i-propoxy, or n-butoxy; or together are 1,2-dioxaethylene, 1,3-dioxapropylene, 1,3-dioxapropylene, 2,3-dimethyl-2,3-dioxabutane, or pinanedioxy.

60. The compound of any one of claims 1, 18, 29, 39 and 56 wherein R¹ and R¹′ are independently hydrogen or a substituted or unsubstituted alkyl group.

61. The compound of any one of claims 1, 18, 29, 39 or 56 wherein R¹ and R¹′ are independently hydrogen or a substituted or unsubstituted alkyl group; provided that R¹ or R^{1′} is not 2,2-difluoroethyl.

62. The compound of any one of claims 1, 18, 29, 39 or 56 wherein R² is —O—(CH₂)_{w}—R⁷ or —(CH₂)_{w}—O—R⁸′.

63. The compound of claim 56 wherein R⁷ or R⁸′ is a substituted or unsubstituted aryl or heteroaryl, wherein the number of substituents, if present, is in the range 1-3.

64. The compound of any one of claims 1, 18, 29, 39 or 56
wherein R² is
2-Pyrazol-1-yl-quinolin-4-olyl,
2-Pyrazol-1-yl-quinazolin-4-olyl,
2-(3-Methyl-pyrazol-1-yl)-quinolin-4-olyl,
2-(3-Methyl-pyrazol-1-yl)-quinazolin-4-olyl,
2-Pyrrolidin-1-yl-quinolin-4-olyl,
2-Pyrrolidin-1-yl-quinazolin-4-olyl,
2-(2-Methylamino-thiazol-4-yl)-quinolin-4-olyl,
2-(2-Methylamino-thiazol-4-yl)-quinazolin-4-olyl,
2-(2,2-Dimethylamino-thiazol-4-yl)-quinolin-4-olyl,
2-(2,2-Dimethylamino-thiazol-4-yl)-quinazolin-4-olyl,
2-(3,3-Difluoro-pyrrolidin-1-yl)-quinolin-4-olyl,
2-(3,3-Difluoro-pyrrolidin-1-yl)-quinazolin-4-olyl,
2-(2,3-Dimethyl-pyrrol-1-yl)-quinolin-4-olyl,
2-(2,3-Dimethyl-pyrrol-1-yl)-quinazolin-4-olyl,
2-Pyrrol-1-yl-quinolin-4-olyl,
2-Pyrrol-1-yl-quinazolin-4-olyl,
2-(3-Methyl-pyrrol-1-yl)-quinolin-4-olyl,
2-(3-Methyl-pyrrol-1-yl)-quinazolin-4-olyl,
2-(2-Isopropylamino-thiazol-4-yl)-quinolin-4-olyl, or
2-(2-Isopropylamino-thiazol-4-yl)-quinazolin-4-olyl.

65. The compound of any one of claims 1, 18, 29, 39 or 56 wherein R⁸′ is attached to a monocyclic heteroaryl group.

66. The compound of any one of claims 1, 18, 29, 39 or 56
wherein:
R⁷ is phenyl, quinolinyl, or quinazolinyl, each optionally substituted with 1-3 substituents selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl, wherein each substituent is optionally substituted with 1 or 2 alkylamino groups.

67. The compound of any one of claims 1 or 56 wherein:
R⁸ is phenyl, quinolinyl, or quinazolinyl, each optionally substituted with 1-3 substituents selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl, wherein each substituent is optionally substituted with 1 or 2 alkylamino groups.

68. The compound of any one of claims 1 or 56 wherein:
R⁸′ is phenyl, quinolinyl, or quinazolinyl, each optionally substituted with 1-3 substituents selected from the group consisting of phenyl, methoxy, pyridinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, quinazolinyl, wherein each substituent is optionally substituted with 1 or 2 alkylamino groups.

69. The compound of any one of claims 1, 18, 29, 39 or 56
wherein:
R⁷ is substituted with 1-3 substituents selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

70. The compound of any one of claims 1 or 56 wherein:
R⁸ is substituted with 1-3 substituents selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

71. The compound of any one of claims 1 or 56 wherein:
R⁸′ is substituted with 1-3 substituents selected from the group consisting of halogen, (C₁₋₁₀)alkyl, (C₁₋₁₀)alkoxy, (C₁₋₁₀)alkylamino, (C₁₋₁₀)dialkylamino, benzyl, benzyloxy, hydroxyl(C₁₋₆)alkyl, hydroxymethyl, nitro, trifluoromethyl, trifluoromethoxy, trifluoromethylthio, N-hydroxyimino, cyano, carboxy, acetamido, hydroxy, sulfamoyl, sulfonamido, and carbamoyl.

72. The compound of any one of claims 1, 18, 29, 39 or 56 wherein R⁴ and R⁵ are hydrogen.

73. The compound of claim 56 wherein R⁶ is hydrogen.

74. The compound of claim 1 wherein:
R⁷′ is phenyl-aminoheteroaryl, quinolinyl-aminoheteroaryl, or quinazolinyl-aminoheteroaryl, each optionally substituted with 1 to 3 substituents selected from methoxy, phenyl, or both.

75. The compound of claim 56 with the structure of Formula Vb:

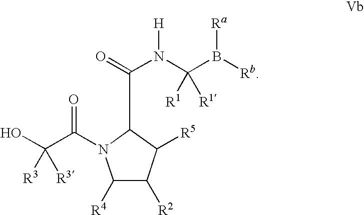

76. The compound of any one of claims 1, 18, 29, 39 or 56 wherein R⁹ is hydrogen.

77. The compound of any one of claims 1, 18, 29, 39 or 56 wherein R³′ is hydrogen.

78. A composition comprising a compound according to any one of claims 1, 18, 29, 39 or 56 and a pharmaceutically acceptable carrier.

79. A method of inhibiting a hepatitis C viral protease comprising contacting said serine protease with a compound of any one of claims 1, 18, 29, 39 or 56 or a composition of claim 78.

80. A method for treating hepatitis C viral infection, comprising administering to a subject in need of such treatment an effective amount of a compound of any one of claims 1, 18, 29, 39 or 56 or a composition of claim 78.
